SEQUENCE LISTING (107 sequences)

SEQ ID NO: 1 - protein, 118 aa, Artificial Sequence
  Description of Artificial Sequence Synthetic polypeptide
QVQLQESGGGLVQAGGSLRLSCAASGTIFDWYSMGWYRQAPGKERELVAGIDTGANTYYADSVKGRFTISRDNAKNTVYLQMNSLKPEDTAVYYCAAHDDGDPWHVYWGQGTQVTVSS

SEQ ID NO: 2 - protein, 122 aa, Artificial Sequence
  Description of Artificial Sequence Synthetic polypeptide
QVQLQESGGGLVQAGGSLRLSCAASGTIFPVDYMGWYRQAPGKERELVAGINYGSNTYYADSVKGRFTISRDNAKNTVYLQMNSLKPEDTAVYYCAAWQPEGYAVDFYHPYWGQGTQVTVSS

SEQ ID NO: 3 - protein, 115 aa, Artificial Sequence
  Description of Artificial Sequence Synthetic polypeptide
QVQLQESGGGLVQAGGSLRLSCAASGSISDRYAMGWYRQAPGKERELVAGIAEGSNTYYADSVKGRFTISRDNAKNTVYLQMNSLKPEDTAVYYCAAHDGWYDYWGQGTQVTVSS

SEQ ID NO: 4 - protein, 111 aa, Artificial Sequence
  Description of Artificial Sequence Synthetic polypeptide
QVQLQESGGGLVQAGGSLRLSCAASGTIFQNLDMGWYRQAPGKERELVAGISYGSSTYYADSVKGRFTISRDNAKNTVYLQMNSLKPEDTAVYYCAVYTYWGQGTQVTVSS

SEQ ID NO: 5 - protein, 119 aa, Artificial Sequence
  Description of Artificial Sequence Synthetic polypeptide
QVQLQESGGGLVQAGGSLRLSCAASGSISRIGDMGWYRQAPGKERELVAAIAAGGTTYYADSVKGRFTISRDNAKNTVYLQMNSLKPEDTAVYYCAASHETQPTQLVYWGQGTQVTVSS

SEQ ID NO: 6 - protein, 122 aa, Artificial Sequence
  Description of Artificial Sequence Synthetic polypeptide
QVQLQESGGGLVQAGGSLRLSCAASGTISPIDIMGWYRQAPGKEREFVAAIALGGNTYYADSVKGRFTISRDNAKNTVYLQMNSLKPEDTAVYYCAVGYVDKWDDSDYHTYWGQGTQVTVSS

SEQ ID NO: 7 - protein, 122 aa, Artificial Sequence
  Description of Artificial Sequence Synthetic polypeptide
QVQLQESGGGLVQAGGSLRLSCAASGSISDWYDMGWYRQAPGKEREFVATIANGSNTYYADSVKGRFTISRDNAKNTVYLQMNSLKPEDTAVYYCAALVGPDDNGWYWLDYWGQGTQVTVSS

SEQ ID NO: 8 - protein, 118 aa, Artificial Sequence
  Description of Artificial Sequence Synthetic polypeptide
QVQLQESGGGLVQAGGSLRLSCAASGNISSISDMGWYRQAPGKERELVAGIGGGANTYYADSVKGRFTISRDNAKNTVYLQMNSLKPEDTAVYYCAAHGYWGWTHEYWGQGTQVTVSS

SEQ ID NO: 9 - protein, 126 aa, Artificial Sequence
  Description of Artificial Sequence Synthetic polypeptide
QVQLQESGGGLVQAGGSLRLSCAASGTISSSADMGWYRQAPGKERELVAGIDRGSNTYYADSVKGRFTISRDNAKNTVYLQMNSLKPEDTAVYYCAAEEVGTGEDDDGADSYHGYWGQGTQVTVSS

SEQ ID NO: 10 - protein, 126 aa, Artificial Sequence
  Description of Artificial Sequence Synthetic polypeptide
QVQLQESGGGLVQAGGSLRLSCAASGTISRDRDMGWYRQAPGKERELVATISPGGTTYYADSVKGRFTISRDNAKNTVYLQMNSLKPEDTAVYYCAAYAAVEEDDSKYYIQDFAYWGQGTQVTVSS

SEQ ID NO: 11 - protein, 126 aa, Artificial Sequence
  Description of Artificial Sequence Synthetic polypeptide
QVQLQESGGGLVQAGGSLRLSCAASGTIFTLPDMGWYRQAPGKEREFVAGIAGGSSTYYADSVKGRFTISRDNAKNTVYLQMNSLKPEDTAVYYCAVGYVAESSDFYDYSNYHEYWGQGTQVTVSS

SEQ ID NO: 12 - protein, 125 aa, Artificial Sequence
  Description of Artificial Sequence Synthetic polypeptide
QVQLQESGGGLVQAGGSLRLSCAASGNISPQHDMGWYRQAPGKERELVATITQGATTYYADSVKGRFTISRDNAKNTVYLQMNSLKPEDTAVYYCAALLYATDPDYVYHVYHVYWGQGTQVTVSS

SEQ ID NO: 13 - protein, 124 aa, Artificial Sequence
  Description of Artificial Sequence Synthetic polypeptide
QVQLQESGGGLVQAGGSLRLSCAASGTIFDYYDMGWYRQAPGKERELVAGISTGTITYYADSVKGRFTISRDNAKNTVYLQMNSLKPEDTAVYYCAAETTSPVVGVDTLWYGYWGQGTQVTVSS

SEQ ID NO: 14 - protein, 123 aa, Artificial Sequence
  Description of Artificial Sequence Synthetic polypeptide
QVQLQESGGGLVQAGGSLRLSCAASGSIFHYYDMGWYRQAPGKERELVATIDPGGTTYYADSVKGRFTISRDNAKNTVYLQMNSLKPEDTAVYYCAAYSTQRNDPETYYLDYWGQGTQVTVSS

SEQ ID NO: 15 - protein, 122 aa, Artificial Sequence
  Description of Artificial Sequence Synthetic polypeptide
QVQLQESGGGLVQAGGSLRLSCAASGYIFQDLDMGWYRQAPGKERELVATITNGGNTNYADSVKGRFTISRDNAKNTVYLQMNSLKPEDTAVYYCAAHFYYVGYGDDEHDYWGQGTQVTVSS

SEQ ID NO: 16 - protein, 122 aa, Artificial Sequence
  Description of Artificial Sequence Synthetic polypeptide
QVQLQESGGGLVQAGGSLRLSCAASGNISSSTDMGWYRQAPGKERELVATISLGGNTYYADSVKGRFTISRDNAKNTVYLQMNSLKPEDTAVYYCAVFEKLGLEDPLYLKYWGQGTQVTVSS

SEQ ID NO: 17 - protein, 120 aa, Artificial Sequence
  Description of Artificial Sequence Synthetic polypeptide
QVQLQESGGGLVQAGGSLSCAASGTIFDWWDMGWYRQAPGKERELVATISYGGNTYYADSVKGRFTISRDNAKNTVYLQMNSLKPEDTAVYYCAVFIPGQWRDYYALTYWGQGTQVTVSS

SEQ ID NO: 18 - protein, 122 aa, Artificial Sequence
  Description of Artificial Sequence Synthetic polypeptide
QVQLQESGGGLVQAGGSLRLSCAASGNISHPAHMGWYRQAPGKEREFVAAIDDGSITYYADSVKGRFTISRDNAKNTVYLQMNSLKPEDTAVYYCAVWQETSVRLGIYFLYWGQGTQVTVSS

SEQ ID NO: 19 - protein, 122 aa, Artificial Sequence
  Description of Artificial Sequence Synthetic polypeptide
QVQLQESGGGLVQAGGSLRLSCAASGSISDGDDMGWYRQAPGKEREFVATIDVGGNTYYADSVKGRFTISRDNAKNTVYLQMNSLKPEDTAVYYCAAAAVDDRDGYYYLLYWGQGTQVTVSS

SEQ ID NO: 20 - protein, 118 aa, Artificial Sequence
  Description of Artificial Sequence Synthetic polypeptide
QVQLQESGGGLVQAGGSLRLSCAASGNIFELYDMGWYRQAPGKERELVAGITYGANTYYADSVKGRFTISRDNAKNTVYLQMNSLKPEDTAVYYCAVHAVNYGYLAYWGQGTQVTVSS

SEQ ID NO: 21 - protein, 117 aa, Artificial Sequence
  Description of Artificial Sequence Synthetic polypeptide
QVQLQESGGGLVQAGGSLRLSCAASGSISAPDDMGWYRQAPGKERELVAGIDLGGNTYYADSVKGRFTISRDNAKNTVYLQMNSLKPEDTAVYYCAAHSTEPPAYGYWGQGTQVTSS

SEQ ID NO: 22 - protein, 118 aa, Artificial Sequence
  Description of Artificial Sequence Synthetic polypeptide
QVQLQESGGGLVQAGGSLRLSCAASGTIFWQVDMGWYRQAPGKERELVAGITSGTNTYYADSVKGRFTISRDNAKNTVYLQMNSLKPEDTAVYYCAAHWPYNQTYTYWGQGTQVTVSS

SEQ ID NO: 23 - protein, 118 aa, Artificial Sequence
  Description of Artificial Sequence Synthetic polypeptide
QVQLQESGGGLVQAGGSLRLSCAASGNIFWYAPMGWYRQAPGKERELVASIADGTSTNYADSVKGRFTISRDNAKNTVYLQMNSLKPEDTAVYYCAAYSEDARDLSYWGQGTQVTVSS

SEQ ID NO: 24 - protein, 118 aa, Artificial Sequence
  Description of Artificial Sequence Synthetic polypeptide
QVQLQESGGGLVQAGGSLRLSCAASGNIFSDFDMGWYRQAPGKERELVAGISVGSNTYYADSVKGRFTISRDNAKNTVYLQMNSLKPEDTAVYYCAAETVKVDYLFYWGQGTQVTVSS

SEQ ID NO: 25 - protein, 118 aa, Artificial Sequence
  Description of Artificial Sequence Synthetic polypeptide
QVQLQESGGGLVQAGGSLRLSCAASGTIFVSGPMGWYRQAPGKEREFVATITDGASTYYADSVKGRFTISRDNAKNTVYLQMNSLKPEDTAVYYCAVADPHDYYHHYWGQGTQVTVSS

SEQ ID NO: 26 - protein, 117 aa, Artificial Sequence
  Description of Artificial Sequence Synthetic polypeptide
QVQLQESGGGLVQAGGSLRLSCAASGNISRYVMGWYRQAPGKERELVAGIDVGAITYYADSVKGRFTISRDNAKNTVYLQMNSLKPEDTAVYYCAVWHYLGYVLAYWGQGTQVTVSS

SEQ ID NO: 27 - protein, 7 aa, Artificial Sequence
  Description of Artificial Sequence Synthetic peptide
TIFDWYS

SEQ ID NO: 28 - protein, 10 aa, Artificial Sequence
  Description of Artificial Sequence Synthetic peptide
LVAGIDTGAN

SEQ ID NO: 29 - protein, 10 aa, Artificial Sequence
  Description of Artificial Sequence Synthetic peptide
AHDDGDPWHV

SEQ ID NO: 30 - protein, 7 aa, Artificial Sequence
  Description of Artificial Sequence Synthetic peptide
SISDRYA

SEQ ID NO: 31 - protein, 10 aa, Artificial Sequence
  Description of Artificial Sequence Synthetic peptide
LVAGIAEGSN

SEQ ID NO: 32 - protein, 7 aa, Artificial Sequence
  Description of Artificial Sequence Synthetic peptide
AHDGWYD

SEQ ID NO: 33 - protein, 7 aa, Artificial Sequence
  Description of Artificial Sequence Synthetic peptide
TIFQNLD

SEQ ID NO: 34 - protein, 10 aa, Artificial Sequence
  Description of Artificial Sequence Synthetic peptide
LVAGISYGSS

SEQ ID NO: 35 - protein, 7 aa, Artificial Sequence
  Description of Artificial Sequence Synthetic peptide
NISSISD

SEQ ID NO: 36 - protein, 10 aa, Artificial Sequence
  Description of Artificial Sequence Synthetic peptide
LVAGIGGGAN

SEQ ID NO: 37 - protein, 10 aa, Artificial Sequence
  Description of Artificial Sequence Synthetic peptide
AHGYWGWTHE

SEQ ID NO: 38 - protein, 7 aa, Artificial Sequence
  Description of Artificial Sequence Synthetic peptide
TIFPVDY

SEQ ID NO: 39 - protein, 10 aa, Artificial Sequence
  Description of Artificial Sequence Synthetic peptide
LVAGINYGSN

SEQ ID NO: 40 - protein, 14 aa, Artificial Sequence
  Description of Artificial Sequence Synthetic peptide
AWQPEGYAVDFYHP

SEQ ID NO: 41 - protein, 7 aa, Artificial Sequence
  Description of Artificial Sequence Synthetic peptide
SISDWYD

SEQ ID NO: 42 - protein, 10 aa, Artificial Sequence
  Description of Artificial Sequence Synthetic peptide
FVATIANGSN

SEQ ID NO: 43 - protein, 14 aa, Artificial Sequence
  Description of Artificial Sequence Synthetic peptide
ALVGPDDNGWYWLD

SEQ ID NO: 44 - protein, 7 aa, Artificial Sequence
  Description of Artificial Sequence Synthetic peptide
TISPIDI

SEQ ID NO: 45 - protein, 10 aa, Artificial Sequence
  Description of Artificial Sequence Synthetic peptide
FVAAIALGGN

SEQ ID NO: 46 - protein, 14 aa, Artificial Sequence
  Description of Artificial Sequence Synthetic peptide
VGYVDKWDDSDYHT

SEQ ID NO: 47 - protein, 7 aa, Artificial Sequence
  Description of Artificial Sequence Synthetic peptide
SISRIGD

SEQ ID NO: 48 - protein, 10 aa, Artificial Sequence
  Description of Artificial Sequence Synthetic peptide
LVAAIAAGGT

SEQ ID NO: 49 - protein, 11 aa, Artificial Sequence
  Description of Artificial Sequence Synthetic peptide
ASHETQPTQLV

SEQ ID NO: 50 - protein, 25 aa, Artificial Sequence
  Description of Artificial Sequence Synthetic peptide
QVQLQESGGGLVQAGGSLRLSCAAS

SEQ ID NO: 51 - protein, 13 aa, Artificial Sequence
  Description of Artificial Sequence Synthetic peptide
MGWYRQAPGKERE

SEQ ID NO: 52 - protein, 40 aa, Artificial Sequence
  Description of Artificial Sequence Synthetic polypeptide
TYYADSVKGRFTISRDNAKNTVYLQMNSLKPEDTAVYYCA

SEQ ID NO: 53 - protein, 12 aa, Artificial Sequence
  Description of Artificial Sequence Synthetic peptide
YWGQGTQVTVSS

SEQ ID NO: 54 - protein, 7 aa, Artificial Sequence
  Description of Artificial Sequence Synthetic peptide
TISSSAD

SEQ ID NO: 55 - protein, 10 aa, Artificial Sequence
  Description of Artificial Sequence Synthetic peptide
LVAGIDRGSN

SEQ ID NO: 56 - protein, 18 aa, Artificial Sequence
  Description of Artificial Sequence Synthetic peptide
AEEVGTGEDDDGADSYHG

SEQ ID NO: 57 - protein, 7 aa, Artificial Sequence
  Description of Artificial Sequence Synthetic peptide
TISRDRD

SEQ ID NO: 58 - protein, 10 aa, Artificial Sequence
  Description of Artificial Sequence Synthetic peptide
LVATISPGGT

SEQ ID NO: 59 - protein, 18 aa, Artificial Sequence
  Description of Artificial Sequence Synthetic peptide
AYAAVEEDDSKYYIQDFA

SEQ ID NO: 60 - protein, 7 aa, Artificial Sequence
  Description of Artificial Sequence Synthetic peptide
TIFTLPD

SEQ ID NO: 61 - protein, 9 aa, Artificial Sequence
  Description of Artificial Sequence Synthetic peptide
VAGIAGGSS

SEQ ID NO: 62 - protein, 18 aa, Artificial Sequence
  Description of Artificial Sequence Synthetic peptide
VGYVAESSDFYDYSNYHE

SEQ ID NO: 63 - protein, 7 aa, Artificial Sequence
  Description of Artificial Sequence Synthetic peptide
NISPQHD

SEQ ID NO: 64 - protein, 10 aa, Artificial Sequence
  Description of Artificial Sequence Synthetic peptide
LVATITQGAT

SEQ ID NO: 65 - protein, 17 aa, Artificial Sequence
  Description of Artificial Sequence Synthetic peptide
ALLYATDPDYVYHVYHV

SEQ ID NO: 66 - protein, 7 aa, Artificial Sequence
  Description of Artificial Sequence Synthetic peptide
TIFDYYD

SEQ ID NO: 67 - protein, 10 aa, Artificial Sequence
  Description of Artificial Sequence Synthetic peptide
LVAGISTGTI

SEQ ID NO: 68 - protein, 16 aa, Artificial Sequence
  Description of Artificial Sequence Synthetic peptide
AETTSPVVGVDTLWYG

SEQ ID NO: 69 - protein, 7 aa, Artificial Sequence
  Description of Artificial Sequence Synthetic peptide
SIFHYYD

SEQ ID NO: 70 - protein, 10 aa, Artificial Sequence
  Description of Artificial Sequence Synthetic peptide
LVATIDPGGT

SEQ ID NO: 71 - protein, 15 aa, Artificial Sequence
  Description of Artificial Sequence Synthetic peptide
AYSTQRNDPETYYLD

SEQ ID NO: 72 - protein, 7 aa, Artificial Sequence
  Description of Artificial Sequence Synthetic peptide
YIFQDLD

SEQ ID NO: 73 - protein, 10 aa, Artificial Sequence
  Description of Artificial Sequence Synthetic peptide
LVATITNGGN

SEQ ID NO: 74 - protein, 14 aa, Artificial Sequence
  Description of Artificial Sequence Synthetic peptide
AHFYYVGYGDDEHD

SEQ ID NO: 75 - protein, 7 aa, Artificial Sequence
  Description of Artificial Sequence Synthetic peptide
NISSSTD

SEQ ID NO: 76 - protein, 10 aa, Artificial Sequence
  Description of Artificial Sequence Synthetic peptide
LVATISLGGN

SEQ ID NO: 77 - protein, 14 aa, Artificial Sequence
  Description of Artificial Sequence Synthetic peptide
VFEKLGLEDPLYLK

SEQ ID NO: 78 - protein, 7 aa, Artificial Sequence
  Description of Artificial Sequence Synthetic peptide
TIFDWWD

SEQ ID NO: 79 - protein, 10 aa, Artificial Sequence
  Description of Artificial Sequence Synthetic peptide
LVATISYGGN

SEQ ID NO: 80 - protein, 14 aa, Artificial Sequence
  Description of Artificial Sequence Synthetic peptide
VFIPGQWRDYYALT

SEQ ID NO: 81 - protein, 7 aa, Artificial Sequence
  Description of Artificial Sequence Synthetic peptide
NISHPAH

SEQ ID NO: 82 - protein, 10 aa, Artificial Sequence
  Description of Artificial Sequence Synthetic peptide
FVAAIDDGSI

SEQ ID NO: 83 - protein, 14 aa, Artificial Sequence
  Description of Artificial Sequence Synthetic peptide
VWQETSVRLGIYFL

SEQ ID NO: 84 - protein, 7 aa, Artificial Sequence
  Description of Artificial Sequence Synthetic peptide
SISDGDD

SEQ ID NO: 85 - protein, 10 aa, Artificial Sequence
  Description of Artificial Sequence Synthetic peptide
FVATIDVGGN

SEQ ID NO: 86 - protein, 14 aa, Artificial Sequence
  Description of Artificial Sequence Synthetic peptide
AAAVDDRDGYYYLL

SEQ ID NO: 87 - protein, 7 aa, Artificial Sequence
  Description of Artificial Sequence Synthetic peptide
NIFELYD

SEQ ID NO: 88 - protein, 10 aa, Artificial Sequence
  Description of Artificial Sequence Synthetic peptide
LVAGITYGAN

SEQ ID NO: 89 - protein, 10 aa, Artificial Sequence
  Description of Artificial Sequence Synthetic peptide
VHAVNYGYLA

SEQ ID NO: 90 - protein, 7 aa, Artificial Sequence
  Description of Artificial Sequence Synthetic peptide
SISAPDD

SEQ ID NO: 91 - protein, 10 aa, Artificial Sequence
  Description of Artificial Sequence Synthetic peptide
LVAGIDLGGN

SEQ ID NO: 92 - protein, 10 aa, Artificial Sequence
  Description of Artificial Sequence Synthetic peptide
AHSTEPPAYG

SEQ ID NO: 93 - protein, 7 aa, Artificial Sequence
  Description of Artificial Sequence Synthetic peptide
TIFWQVD

SEQ ID NO: 94 - protein, 10 aa, Artificial Sequence
  Description of Artificial Sequence Synthetic peptide
LVAGITSGTN

SEQ ID NO: 95 - protein, 10 aa, Artificial Sequence
  Description of Artificial Sequence Synthetic peptide
AHWPYNQTYT

SEQ ID NO: 96 - protein, 7 aa, Artificial Sequence
  Description of Artificial Sequence Synthetic peptide
NIFWYAP

SEQ ID NO: 97 - protein, 10 aa, Artificial Sequence
  Description of Artificial Sequence Synthetic peptide
LVASIADGTS

SEQ ID NO: 98 - protein, 10 aa, Artificial Sequence
  Description of Artificial Sequence Synthetic peptide
AYSEDARDLS

SEQ ID NO: 99 - protein, 7 aa, Artificial Sequence
  Description of Artificial Sequence Synthetic peptide
NIFSDFD

SEQ ID NO: 100 - protein, 10 aa, Artificial Sequence
  Description of Artificial Sequence Synthetic peptide
LVAGISVGSN

SEQ ID NO: 101 - protein, 10 aa, Artificial Sequence
  Description of Artificial Sequence Synthetic peptide
AETVKVDYLF

SEQ ID NO: 102 - protein, 7 aa, Artificial Sequence
  Description of Artificial Sequence Synthetic peptide
TIFVSGP

SEQ ID NO: 103 - protein, 10 aa, Artificial Sequence
  Description of Artificial Sequence Synthetic peptide
FVATITDGAS

SEQ ID NO: 104 - protein, 10 aa, Artificial Sequence
  Description of Artificial Sequence Synthetic peptide
VADPHDYYHH

SEQ ID NO: 105 - protein, 6 aa, Artificial Sequence
  Description of Artificial Sequence Synthetic peptide
NISRYV

SEQ ID NO: 106 - protein, 10 aa, Artificial Sequence
  Description of Artificial Sequence Synthetic peptide
LVAGIDVGAI

SEQ ID NO: 107 - protein, 10 aa, Artificial Sequence
  Description of Artificial Sequence Synthetic peptide
VWHYLGYVLA

(19) United States
(12) Patent Application Publication
(10) Pub. No.: US 2022/0251217 A1
(43) Pub. Date: Aug. 11, 2022

(54) IMMUNOTHERAPY TARGETING CELL SURFACE MARKER CD72 FOR THE TREATMENT OF B-CELL MALIGNANCIES

(71) Applicant: The Regents of the University of California, Oakland, CA (US)

(72) Inventors: Matthew Nix, Oakland, CA (US); Arun Wiita, Oakland, CA (US)

(21) Appl. No.: 17/623,735

(22) PCT Filed: Jul. 2, 2020
(86) PCT No.: PCT/US2020/040749

§ 371 Date: Dec. 29, 2021

Related U.S. Application Data
(60) Provisional application No. 62/870,463, filed on Jul. 3, 2019.

(51) Int. Cl. C07K 16/28; C12N 5/0783; A61K 35/17; A61P 35/02

(52) U.S. Cl. CPC C07K 16/2851; C12N 5/0638; A61K 35/17; A61P 35/02; C07K 2317/92; C07K 2317/565; C07K 2317/21; C07K 2319/03; C07K 2319/33; C07K 2317/569

(57) ABSTRACT

Provided herein are anti-CD72 nanobodies and methods of using such nanobodies for diagnostic and therapeutic purposes.

### CROSS-REFERENCE TO RELATED APPLICATIONS

This application claims priority benefit of U.S. provisional application No. 62/870,463, filed Jul. 3, 2019, which is incorporated by reference herein for all purposes.

### STATEMENT AS TO RIGHTS TO INVENTIONS MADE UNDER FEDERALLY SPONSORED RESEARCH AND DEVELOPMENT

This invention was made with government support under grants no. K08 CA184116 and OD022552 awarded by the National Institutes of Health. The government has certain rights in the invention.

### BACKGROUND OF THE INVENTION

Despite the enormous strides made in the treatment of hematological malignancies, many patients continue to experience poor clinical outcomes. In 2020 alone, its estimated that nearly 178,000 people will be diagnosed with a blood cancer in the United States while >56,000 people will die. (Cancer Facts & Figures, 2020. American Cancer Society; 2020). Although development of new immunotherapies such as CD19 or CD22-directed CAR-T have achieved remarkable initial response rates, a large fraction of these patients eventually relapse. Therefore, new strategies are needed for the treatment of refractory B-cell malignancies, and in particular alternative surface targets to direct CAR-T cells.

### BRIEF SUMMARY OF ASPECTS OF THE DISCLOSURE

Using cell-surface proteomics of B-ALL cell lines and analysis of patient samples to identify cell-surface markers specifically enriched on B-ALL, CD72 was identified as a potential alternative immunotherapy target, orthogonal and complimentary to current CD19 and CD22 directed therapies. CD72 is a highly abundant cell surface protein found enriched on leukemia and lymphoma cells, similar to the canonical B-cell markers CD19 and CD22 that are currently being targeted with immunotherapies in the clinic. Accordingly, provided herein are anti-CD72 nanobodies that can be used for diagnostic and therapeutic purposes, e.g., for the development of CAR-T therapies that target CD72-expressing malignancies.

In one aspect, provided herein is a nanobody that specifically binds to CD72, wherein the nanobody comprises:

(a) a CDR1 sequence comprising TIFDWYS, a CDR2 sequence comprising LVAGIDTGAN, and a CDR3 sequence comprising AHDDGDPWHV;
(b) a CDR1 sequence comprising SISDRYA, a CDR2 sequence comprising LVAGIAEGSN, and a CDR3 sequence comprising AHDGWYD;
(c) a CDR1 sequence comprising TIFQNLD, a CDR2 sequence comprising LVAGISYGSS, and a CDR3 sequence comprising VYT;
(d) a CDR1 sequence comprising NISSISD, a CDR2 sequence comprising LVAGIGGGAN, and a CDR3 sequence comprising AHGYWGWTHE;
(e) a CDR1 sequence comprising TIFPVDY, a CDR2 sequence comprising LVAGINYGSN, and a CDR3 sequence comprising AWQPEGYAVDFYHP;
(f) a CDR1 sequence comprising SISDWYD, a CDR2 sequence comprising FVATIANGSN, and a CDR3 sequence comprising ALVGPDDNGWYWLD;
(g) a CDR1 sequence comprising TISPIDI, a CDR2 sequence comprising FVAAIALGGN, and a CDR3 sequence comprising VGYVDKWDDSDYHT; or
(h) a CDR1 sequence comprising SISRIGD, a CDR2 sequence comprising LVAAIAAGGT, and a CDR3 sequence comprising ASHETQPTQLV.

In some embodiments, the nanobody comprises:

(a) the CDR1 sequence comprising SISRIGD, the CDR2 sequence comprising LVAAIAAGGT, and the CDR3 sequence comprising ASHETQPTQLV;
(b) the CDR1 sequence comprising TISPIDI, the CDR2 sequence comprising FVAAIALGGN, and the CDR3 sequence comprising VGYVDKWDDSDYHT; or
(c) the CDR1 sequence comprising TIFQNLD, the CDR2 sequence comprising LVAGISYGSS, and the CDR3 sequence comprising VYT.

In some embodiments, the nanobody comprises the CDR1 sequence comprising TISPIDI, the CDR2 sequence comprising FVAAIALGGN, and the CDR3 sequence comprising VGYVDKWDDSDYHT. In some embodiments, the framework has at least 80% identity to a human antibody heavy chain framework, e.g., a VH3 family member. In some embodiments, the nanobody comprises a framework having at least 80%, or at least 85%, at least 90%, or at least 95%, identity to a framework of comprising an FR1 sequence QVQLQESGGGLVQAGGSLRLSCAAS, an FR2 sequence MGWYRQAPGKERE, an FR3 sequence TYYADSVKGRFTISRDNAKNTVYLQMNSLKPEDTAVYYCA, and an FR4 sequence YWGQGTQVTVSS.

Additionally provided herein is a nanobody that specifically binds to CD72, wherein the nanobody comprises:

(a) a CDR1 sequence comprising TIFDWYS, a CDR2 sequence comprising LVAGIDTGAN, and a CDR3 sequence comprising AHDDGDPWHV in which at least one of the CDR1, CDR2, or CDR3 has 1 or 2 amino acid substitutions;
(b) a CDR1 sequence comprising SISDRYA, a CDR2 sequence comprising LVAGIAEGSN, and a CDR3 sequence comprising AHDGWYD in which at least one of the CDR1, CDR2, or CDR3 has 1 or 2 amino acid substitutions;
(c) a CDR1 sequence comprising TIFQNLD, a CDR2 sequence comprising LVAGISYGSS, and a CDR3 sequence comprising VYT in which at least one of the CDR1, CDR2, or CDR3 has 1 or 2 amino acid substitutions;
(d) a CDR1 sequence comprising NISSISD, a CDR2 sequence comprising LVAGIGGGAN, and a CDR3 sequence comprising AHGYWGWTHE in which at least one of the CDR1, CDR2, or CDR3 has 1 or 2 amino acid substitutions;
(e) a CDR1 sequence comprising TIFPVDY, a CDR2 sequence comprising LVAGINYGSN, and a CDR3 sequence comprising AWQPEGYAVDFYHP in which at least one of the CDR1, CDR2, or CDR3 has 1 or 2 amino acid substitutions;
(f) a CDR1 sequence comprising SISDWYD, a CDR2 sequence comprising FVATIANGSN, and a CDR3 sequence comprising ALVGPDDNGWYWLD in which at least one of the CDR1, CDR2, or CDR3 has 1 or 2 amino acid substitutions;
(g) a CDR1 sequence comprising TISPIDI, a CDR2 sequence comprising FVAAIALGGN, and a CDR3 sequence comprising VGYVDKWDDSDYHT in which at least one of the CDR1, CDR2, or CDR3 has 1 or 2 amino acid substitutions; or
(h) a CDR1 sequence comprising SISRIGD, a CDR2 sequence comprising LVAAIAAGGT, and a CDR3 sequence comprising ASHETQPTQLV in which at least one of the CDR1, CDR2, or CDR3 has 1 or 2 amino acid substitutions.

Additionally provided herein is a nanobody that specifically binds to CD72, wherein the nanobody comprises:
(a) a CDR1 sequence comprising TISSSAD, a CDR2 sequence comprising LVAGIDRGSN, and a CDR3 sequence comprising AEEVGTGEDDDGADSYHG; or a variant thereof in which at least one of the CDRs has 1 or 2 amino acid substitutions;
(b) a CDR1 sequence comprising TISRDRD, a CDR2 sequence comprising LVATISPGGT, and a CDR3 sequence comprising AYAAVEEDDSKYYIQDFA; or a variant thereof in which at least one of the CDRs has 1 or 2 amino acid substitutions;
(c) a CDR1 sequence comprising TIFTLPD, a CDR2 sequence comprising VAGIAGGSS, and a CDR3 sequence comprising VGYVAESSDFYDYSNYHE; or a variant thereof in which at least one of the CDRs has 1 or 2 amino acid substitutions;
(d) a CDR1 sequence comprising NISPQHD, a CDR2 sequence comprising LVATITQGAT, and a CDR3 sequence comprising ALLYATDPDYVYHVYHV; or a variant thereof in which at least one of the CDRs has 1 or 2 amino acid substitutions;
(e) a CDR1 sequence comprising TIFDYYD, a CDR2 sequence comprising LVAGISTGTI, and a CDR3 sequence comprising AETTSPVVGVDTLWYG; or a variant thereof in which at least one of the CDRs has 1 or 2 amino acid substitutions;
(f) a CDR1 sequence comprising SIFHYYD, a CDR2 sequence comprising LVATIDPGGT, and a CDR3 sequence comprising AYSTQRNDPETYYLD; or a variant thereof in which at least one of the CDRs has 1 or 2 amino acid substitutions;
(g) a CDR1 sequence comprising YIFQDLD, a CDR2 sequence comprising LVATITNGGN, and a CDR3 sequence comprising AHFYYVGYGDDEHD; or a variant thereof in which at least one of the CDRs has 1 or 2 amino acid substitutions;
(h) a CDR1 sequence comprising NISSSTD, a CDR2 sequence comprising LVATISLGGN, and a CDR3 sequence comprising VFEKLGLEDPLYLK; or a variant thereof in which at least one of the CDRs has 1 or 2 amino acid substitutions;
(i) a CDR1 sequence comprising TIFDWWD, a CDR2 sequence comprising LVATISYGGN, and a CDR3 sequence comprising VFIPGQWRDYYALT; or a variant thereof in which at least one of the CDRs has 1 or 2 amino acid substitutions;
(j) a CDR1 sequence comprising NISHPAH, CDR2 sequence comprising FVAAIDDGSI, a CDR3 sequence comprising VWQETSVRLGIYFL; or a variant thereof in which at least one of the CDRs has 1 or 2 amino acid substitutions;
(k) a CDR1 sequence comprising SISDGDD, a CDR2 sequence comprising FVATIDVGGN, and a CDR3 sequence comprising AAAVDDRDGYYYLL; or a variant thereof in which at least one of the CDRs has 1 or 2 amino acid substitutions;
(l) a CDR1 sequence comprising NIFELYD, a CDR2 sequence comprising LVAGITYGAN, and a CDR3 sequence comprising VHAVNYGYLA; or a variant thereof in which at least one of the CDRs has 1 or 2 amino acid substitutions;
(m) a CDR1 sequence comprising SISAPDD, a CDR2 sequence comprising LVAGIDLGGN, and a CDR3 sequence comprising AHSTEPPAYG; or a variant thereof in which at least one of the CDRs has 1 or 2 amino acid substitutions;
(n) a CDR1 sequence comprising TIFWQVD, a CDR2 sequence comprising LVAGITSGTN, and a CDR3 sequence comprising AHWPYNQTYT; or a variant thereof in which at least one of the CDRs has 1 or 2 amino acid substitutions;
(o) a CDR1 sequence comprising NIFWYAP, a CDR2 sequence comprising LVASIADGTS, and a CDR3 sequence comprising AYSEDARDLS; or a variant thereof in which at least one of the CDRs has 1 or 2 amino acid substitutions;
(p) a CDR1 sequence comprising NIFSDFD, a CDR2 sequence comprising LVAGISVGSN, and a CDR3 sequence comprising AETVKVDYLF; or a variant thereof in which at least one of the CDRs has 1 or 2 amino acid substitutions;
(q) a CDR1 sequence comprising TIFVSGP, a CDR2 sequence comprising FVATITDGAS, and a CDR3 sequence comprising VADPHDYYHH; or a variant thereof in which at least one of the CDRs has 1 or 2 amino acid substitutions; or
(r) a CDR1 sequence comprising NISRYV, a CDR2 sequence comprising LVAGIDVGAI, and a CDR3 sequence comprising VWHYLGYVLA; or a variant thereof in which at least one of the CDRs has 1 or 2 amino acid substitutions.
   In some embodiments, the antibody comprises a variable region comprising:
(a) a CDR1 sequence comprising TISSSAD, a CDR2 sequence comprising LVAGIDRGSN, and a CDR3 sequence comprising AEEVGTGEDDDGADSYHG;
(b) a CDR1 sequence comprising TISRDRD, a CDR2 sequence comprising LVATISPGGT, and a CDR3 sequence comprising AYAAVEEDDSKYYIQDFA;
(c) a CDR1 sequence comprising TIFTLPD, a CDR2 sequence comprising VAGIAGGSS, and a CDR3 sequence comprising VGYVAESSDFYDYSNYHE;
(d) a CDR1 sequence comprising NISPQHD, a CDR2 sequence comprising LVATITQGAT, and a CDR3 sequence comprising ALLYATDPDYVYHVYHV;
(e) a CDR1 sequence comprising TIFDYYD, a CDR2 sequence comprising LVAGISTGTI, and a CDR3 sequence comprising AETTSPVVGVDTLWYG;
(f) a CDR1 sequence comprising SIFHYYD, a CDR2 sequence comprising LVATIDPGGT, and a CDR3 sequence comprising AYSTQRNDPETYYLD;
(g) a CDR1 sequence comprising YIFQDLD, a CDR2 sequence comprising LVATITNGGN, and a CDR3 sequence comprising AHFYYVGYGDDEHD;
(h) a CDR1 sequence comprising NISSSTD, a CDR2 sequence comprising LVATISLGGN, and a CDR3 sequence comprising VFEKLGLEDPLYLK;
(i) a CDR1 sequence comprising TIFDWWD, a CDR2 sequence comprising LVATISYGGN, and a CDR3 sequence comprising VFIPGQWRDYYALT;
(j) a CDR1 sequence comprising NISHPAH, CDR2 sequence comprising FVAAIDDGSI, a CDR3 sequence comprising VWQETSVRLGIYFL;
(k) a CDR1 sequence comprising SISDGDD, a CDR2 sequence comprising FVATIDVGGN, and a CDR3 sequence comprising AAAVDDRDGYYYLL;
(l) a CDR1 sequence comprising NIFELYD, a CDR2 sequence comprising LVAGITYGAN, and a CDR3 sequence comprising VHAVNYGYLA;
(m) a CDR1 sequence comprising SISAPDD, a CDR2 sequence comprising LVAGIDLGGN, and a CDR3 sequence comprising AHSTEPPAYG;
(n) a CDR1 sequence comprising TIFWQVD, a CDR2 sequence comprising LVAGITSGTN, and a CDR3 sequence comprising AHWPYNQTYT;
(o) a CDR1 sequence comprising NIFWYAP, a CDR2 sequence comprising LVASIADGTS, and a CDR3 sequence comprising AYSEDARDLS;
(p) a CDR1 sequence comprising NIFSDFD, a CDR2 sequence comprising LVAGISVGSN, and a CDR3 sequence comprising AETVKVDYLF;
(q) a CDR1 sequence comprising TIFVSGP, a CDR2 sequence comprising FVATITDGAS, and a CDR3 sequence comprising VADPHDYYHH; or
(r) a CDR1 sequence comprising NISRYV, a CDR2 sequence comprising LVAGIDVGAI, and a CDR3 sequence comprising VWHYLGYVLA.

In a further aspect, provided herein is a chimeric antigen receptor (CAR) comprising an antigen binding domain, a transmembrane domain, and an intracellular domain comprising a costimulatory domain and/or a primary signaling domain, wherein the antigen binding domain comprises an anti-CD72 nanobody as described herein, e.g., in the preceding paragraphs in this section. In some embodiments, the CAR comprises an antigen binding domain, a transmembrane domain, and a cytoplasmic signaling domain comprising a costimulatory domain and/or a primary signaling domain, wherein the antigen binding domain comprises a nanobody comprising:

(a) the CDR1 sequence comprising SISRIGD, the CDR2 sequence comprising LVAAIAAGGT, and the CDR3 sequence comprising ASHETQPTQLV;
(b) the CDR1 sequence comprising TISPIDI, the CDR2 sequence comprising FVAAIALGGN, and the CDR3 sequence comprising VGYVDKWDDSDYHT; or
(c) the CDR1 sequence comprising TIFQNLD, the CDR2 sequence comprising LVAGISYGSS, and the CDR3 sequence comprising VYT.
In some embodiments, the antigen binding domain comprises two, three or four nanobodies selected from the group consisting of:
(a) a nanobody comprising a CDR1 sequence comprising TIFDWYS, a CDR2 sequence comprising LVAGIDTGAN, and a CDR3 sequence comprising AHDDGDPWHV;
(b) a nanobody comprising a CDR1 sequence comprising SISDRYA, a CDR2 sequence comprising LVAGIAEGSN, and a CDR3 sequence comprising AHDGWYD;
(c) a nanobody comprising a CDR1 sequence comprising TIFQNLD, a CDR2 sequence comprising LVAGISYGSS, and a CDR3 sequence comprising VYT;
(d) a nanobody comprising a CDR1 sequence comprising NISSISD, a CDR2 sequence comprising LVAGIGGGAN, and a CDR3 sequence comprising AHGYWGWTHE;
(e) a nanobody comprising a CDR1 sequence comprising TIFPVDY, a CDR2 sequence comprising LVAGINYGSN, and a CDR3 sequence comprising AWQPEGYAVDFYHP;
(f) a nanobody comprising a CDR1 sequence comprising SISDWYD, a CDR2 sequence comprising FVATIANGSN, and a CDR3 sequence comprising ALVGPDDNGWYWLD;
(g) a nanobody comprising a CDR1 sequence comprising TISPIDI, a CDR2 sequence comprising FVAAIALGGN, and a CDR3 sequence comprising VGYVDKWDDSDYHT; and
(h) a nanobody comprising a CDR1 sequence comprising SISRIGD, a CDR2 sequence comprising LVAAIAAGGT, and a CDR3 sequence comprising ASHETQPTQLV.
In some embodiments, the antigen binding domain comprises one, two or three nanobodies selected from the group consisting of
(a) a nanobody comprising the CDR1 sequence comprising SISRIGD, the CDR2 sequence comprising LVAAIAAGGT, and the CDR3 sequence comprising ASHETQPTQLV;
(b) a nanobody comprising the CDR1 sequence comprising TISPIDI, the CDR2 sequence comprising FVAAIALGGN, and the CDR3 sequence comprising VGYVDKWDDSDYHT; and
(c) a nanobody comprising the CDR1 sequence comprising TIFQNLD, the CDR2 sequence comprising LVAGISYGSS, and the CDR3 sequence comprising VYT.
In some embodiments, the CAR is a standard CAR, a split CAR, an off-switch CAR, an on-switch CAR, a first-generation CAR, a second-generation CAR, a third-generation CAR, or a fourth-generation CAR.

In a further aspect, provided herein is a synthetic Notch receptor comprising at least one anti-CD72 nanobody that comprises:

(a) the CDR1 sequence comprising SISRIGD, the CDR2 sequence comprising LVAAIAAGGT, and the CDR3 sequence comprising ASHETQPTQLV;
(b) the CDR1 sequence comprising TISPIDI, the CDR2 sequence comprising FVAAIALGGN, and the CDR3 sequence comprising VGYVDKWDDSDYHT; or
(c) the CDR1 sequence comprising TIFQNLD, the CDR2 sequence comprising LVAGISYGSS, and the CDR3 sequence comprising VYT. In some embodiments, the antigen binding domain comprises two, three or four nanobodies selected from the group consisting of:
(a) a nanobody comprising a CDR1 sequence comprising TIFDWYS, a CDR2 sequence comprising LVAGIDTGAN, and a CDR3 sequence comprising AHDDGDPWHV;
(b) a nanobody comprising a CDR1 sequence comprising SISDRYA, a CDR2 sequence comprising LVAGIAEGSN, and a CDR3 sequence comprising AHDGWYD;
(c) a nanobody comprising a CDR1 sequence comprising TIFQNLD, a CDR2 sequence comprising LVAGISYGSS, and a CDR3 sequence comprising VYT;
(d) a nanobody comprising a CDR1 sequence comprising NISSISD, a CDR2 sequence comprising LVAGIGGGAN, and a CDR3 sequence comprising AHGYWGWTHE;
(e) a nanobody comprising a CDR1 sequence comprising TIFPVDY, a CDR2 sequence comprising LVAGINYGSN, and a CDR3 sequence comprising AWQPEGYAVDFYHP;
(f) a nanobody comprising a CDR1 sequence comprising SISDWYD, a CDR2 sequence comprising FVATIANGSN, and a CDR3 sequence comprising ALVGPDDNGWYWLD;
(g) a nanobody comprising a CDR1 sequence comprising TISPIDI, a CDR2 sequence comprising FVAAIALGGN, and a CDR3 sequence comprising VGYVDKWDDSDYHT; and
(h) a nanobody comprising a CDR1 sequence comprising SISRIGD, a CDR2 sequence comprising LVAAIAAGGT, and a CDR3 sequence comprising ASHETQPTQLV.
In some embodiments, the antigen binding domain of the synthetic Notch receptor comprises one, two or three nanobodies selected from the group consisting of
(a) a nanobody comprising the CDR1 sequence comprising SISRIGD, the CDR2 sequence comprising LVAAIAAGGT, and the CDR3 sequence comprising ASHETQPTQLV;
(b) a nanobody comprising the CDR1 sequence comprising TISPIDI, the CDR2 sequence comprising FVAAIALGGN, and the CDR3 sequence comprising VGYVDKWDDSDYHT; and
(c) a nanobody comprising the CDR1 sequence comprising TIFQNLD, the CDR2 sequence comprising LVAGISYGSS, and the CDR3 sequence comprising VYT.

In a further aspect, provided herein is an immune effector cell comprising a CAR or synthetic Notch receptor comprising one or more anti-CD72 nanobodies as described herein, e.g., as described in the preceding paragraphs. In some embodiments, the immune effector cell is a T lymphocyte or a natural killer (NK) cell. In some embodiments, the immune effector cell is an autologous cell from a subject to be treated with the immune effector cell. In other embodiments, the immune effector cell is an allogeneic cell.

In a further aspect, provided herein is a method of treating a hematological malignancy that comprises malignant B cells that express CD72 or a malignancy that comprises malignant myeloid cells that express CD72, the method comprising administering a plurality of immune effector cells genetically modified to express one or more anti-CD72 nanobodies as described herein to a subject that has the hematological malignancy. In some embodiments, the hematological malignancy is a B-cell leukemia, e.g., chronic lymphocytic leukemia. In some embodiments, the hematological malignancy mixed-lineage leukemia (MLL). In some embodiments, the hematological malignancy is a non-Hodgkin's lymphoma. In some embodiments, the hematological malignancy is multiple myeloma.

Additionally provided herein is a polynucleotide encoding an anti-CD72 nanobody as described here. In further embodiments, the disclosure provides a polynucleotide encoding a CAR comprising one or more anti-CD72 nanobody of the present invention. Further, the disclosure provides vectors comprising such polynucleotides and mammalian host cells, e.g., immune effector cells, comprising the polynucleotides. In some embodiments, the vector a retroviral vector, e.g., a self-inactivating lentiviral vector. In some embodiments, the immune effector cell is a T lymphocyte or NK cell.

### BRIEF DESCRIPTION OF THE DRAWINGS

FIG. 1a-d: Multi-omics analysis of the MLLr B-ALL cell surfaceome uncovers unique cell surface signatures and survival dependencies. (a) Proteomics workflow for quantifying the cell surfaceomes of B-ALL cell lines. (b) Volcano plot displaying MLLr upregulated cell surface proteins. The log 2-fold change comparing the label-free quantification values (LFQ) of MLLr versus non-MLLr cell lines is plotted on the x-axis, while the −log 10(p-value) is plotted on the y-axis. Proteins with log 2-fold change >2 and −log 10(p-value) >1.3 were considered significantly upregulated, with select proteins labeled. Significance and upregulation cut-offs are shown by dotted lines. Statistical analysis conducted using a two-sided Welch's T-test. (c) Principal component analysis of the B-ALL cell surfaceome. (d) Volcano plot displaying MLLr upregulated transcripts of cell surface proteins. The log 2-fold change of the FPKM of different transcripts is shown on the x-axis while the −log 10(p-value) is shown on the y-axis. Upregulated transcripts (log 2-fold >2 and −log 10(p-value)>1.3) are shown in blue with select genes labeled. Genes identified through proteomics as up or down regulated, but were missed by transcriptome analysis are shown in orange and are labeled. Statistical analysis conducted using a two-sided Welch's T-test.

FIG. 2a-g: CD72 is a highly-abundant receptor on the cell surface of MLLr B-ALL and other B-cell malignancies. (a) Schematic showing triage of cell surface membrane proteins to identify immunotherapy candidates for MLLr B-ALL. (b). Transcript abundance of immunotherapy targets CD22, CD19, and immunotherapy candidate CD72, in 29 different immune cell types measured by RNAseq (Human Protein Atlas Database, GSE107011, (www website proteinatlas.org). (c) Median normal tissue transcript abundance of CD72 according to GTex RNAseq data (Log 2 TPM, datafile GTEx_Analysis_2016-01-15_v7_RNASeQCv1.1.8_gene_median_tpm.gct). (d) Transcript abundance of CD72 in malignant cell lines (n=1461; CCLE, accessed Oct. 14, 2019). TPM, transcript per million mapped reads. (e) Transcript abundance of CD72 in normal and malignant patient samples (ECOG E2993, n=191, GSE34861), (COG P9906, n=207, GSE11877), (St. Jude, n=132, www website stjuderesearch.org/data/ALL3), (St. Jude, n=154, GSE26281). y-axis shows log 2-transformed abundance by microarray gene expression. (f) Plot comparing the log 2 transcript abundance of CD22, CD72, and CD19 by microarray analysis of a DLBCL patient cohort (GSE12195, n=73). (g) CD72 transcript abundance by microarray analysis of ABC and GCB subtypes of DLBCL patient samples (GSE11318, n=203 and GSE23967, n=69).

FIG. 3a-f: Quantification of CD72 abundance in B-ALL and DLBCL via flow cytometry and immunohistochemistry (a) Flow cytometry histograms of CD72 and CD19 surface density on MLLr B-ALL patient-derived xenografts and cell lines. Molecules of receptor per cell were calculated using a quantitative flow cytometry assay. (b) Representative flow cytometry histograms of CD72 surface density on viably-frozen, pediatric B-ALL patient samples. Log 2 of the Median Fluorescence Intensity (MFI) of CD72 staining is graphed on the y-axis, comparing MLLr to non-MLLr patient samples on the x-axis (total, n=11) (c) Quantification of CD72 abundance by immunohistochemistry (IHC) staining of banked adult B-ALL patient bone marrow aspirates (total, n=15). Each tumor was graded for staining percentage and intensity by two independent pathologists blinded to sample identity, which were used to calculate IHC H-scores (range: 0-300). (d) Representative raw images of CD72 staining intensity by IHC of two different B-ALL subtypes. (e) Quantification of CD72 abundance by immunohistochemistry (IHC) staining of banked DLBCL patient samples (total, n=28) displayed by ABC or GCB subtype. (f) Representative raw images of CD72 staining intensity by IHC of two different DLBCL patient samples.

FIG. 4a-e: Isolation of high-affinity CD72 nanobodies with yeast display (a) Schematic of workflow for in vitro anti-CD72 nanobody selection using yeast display. (b) Structure models of the recombinant Fc-fusion proteins used to perform yeast display selections. The Fc protein on the left was used to negatively select potential off-target nanobodies while the CD72-Fc protein (CD72 extracellular domain fused to a human Fc domain) was used to perform positive selection steps to isolate CD72-specific nanobodies (c) Schematic displaying the nanobody yeast display selection strategy for each MACS and FACS selection round to enrich for CD72-specific nanobody binders. Two rounds of MACS followed by four rounds of FACS with decreasing concentration of CD72 antigen produced high affinity anti-CD72 nanobodies. (d) On-yeast binding of recombinant CD72-Fc fusion protein for CD72-selected nanobodies Nb.B5 (nanobody sequence SEQ ID NO:2) and Nb.C2 (nanobody sequence SEQ ID NO:1) expressed on yeast to determine estimated binding affinities. Kd determined by curve fitting using non-linear least squares regression. (e) Flow cytometry plots of yeast clone Nb.C2 binding to 10 nM CD72-ECD-Fc protein (left) or 10 nM Fc protein (right). Y-axis displays the anti-biotin-APC signal (corresponding to yeast binding to recombinant protein) and the x-axis displays the anti-HA-FITC signal (corresponding to nanobodies displayed on the yeast surface).

FIG. 5a-f: Nanobody-based CD72 CAR T's demonstrate potent in vitro cytotoxicity against B-ALL cell lines (a) CD72-directed nanobody sequences were incorporated into a second-generation CAR backbone design including a CD8 hinge and transmembrane domain (TM), 4-1BB co-stimulatory domain, and CD3; activation domain. (b) Jurkat activation assay measuring antigen-dependent and independent signaling of eight candidate nanobody CAR constructs. Jurkat-CAR's were incubated overnight (1:1 ratio) with either the CD72-negative cell line AMO1 (antigen-independent) or the CD72-positive cell line RS411 (antigen-dependent). Activation measured by CD69 mean fluorescence intensity (MFI) normalized to isotype control MFI (left side y-axis). The ratio of antigen-dependent over antigen-independent MFI is shown with a circle (right y-axis). (c) In vitro cytotoxicity of CD72 CAR-T clones versus SEM or RS411 cell lines. Cytotoxicity measured using DRAQ7 stain after 24-hour co-culture at a 1:1 ratio. FACS plots show the percent of DRAQ7+ cells in single point experiments. Barplots show the percent cytotoxicity of CD72 CAR-T clones normalized to CD19 CAR-T cytotoxicity. (d-f) In vitro cytotoxicity of 18 different CD72 CAR-T's cocultured with the SEM cell line (labeled with firefly luciferase) at various effector to target ratios for 8 hours. The y-axis shows percent specific lysis while the x-axis shows the ratio of effector to target. Cytotoxicity measured using bioluminescence. Experiments were performed in triplicate and signals were normalized to control wells containing only target cells. Data represented by mean+/−SEM. Equivalents of effector cells were adjusted to account for the % of CAR+ cells.

FIG. 6a-d: in vitro cytotoxicity of CD72(Nb.D4) CAR-T against multiple B-cell malignancies Cytotoxicity of CD72 (Nb.D4), CD19, or empty CAR T against leukemia and lymphoma cell lines at varying effector:target ratios, cocultured for 4 hrs. (a) Cytotoxicity versus the SEM cell line (B-ALL). (b) Cytotoxicity versus the JEKO-1 cell line (Mantle Cell Lymphoma). (c) Cytotoxicity versus the Namalwa cell line (Burkitt Lymphoma). (d) Cytotoxicity versus the HBL1 cell line (DLBCL). All target cells stably expressed enhanced-firefly luciferase to enable viability measurements with bioluminescence imaging. Experiments were performed in triplicate and signals were normalized to control wells containing only target cells. Data represented by mean+/−SEM. Equivalents of effector cells were adjusted to account for the % of CAR+ cells.

FIG. 7a-c: in vitro cytotoxicity of CD72(Nb.D4) CAR-T against gene-edited B-ALL cell lines Cytotoxicity of CD72 (Nb.D4), CD19, or empty CAR T against parental or gene edited SEM cell lines at varying effector:target ratios, cocultured for 48 hrs. (a) Cytotoxicity versus wt SEM cells. (b) Cytotoxicity versus CD19-knockdown CRISPRi-edited SEM cells. (c) Cytotoxicity versus CD72-knockdown CRISPRi-edited SEM cells. All target cells stably expressed enhanced-firefly luciferase to enable viability measurements with bioluminescence imaging. Experiments were performed in triplicate and signals were normalized to control wells containing only target cells. Data represented by mean+/−SEM. Equivalents of effector cells were adjusted to account for the % of CAR+ cells.

FIG. 8a-c: CD72 CAR T eradicates tumors and prolongs survival in cell line and xenograft models of B-ALL NSG mice were injected with 1e6 firefly-luciferase labeled tumor cells including an MLLr B-ALL patient-derived xenograft, the parental SEM MLLr B-ALL cell line, and a CD19-knockdown CRISPRi SEM cell line (CD19-MLLr B-ALL). After confirming engraftment, mice were treated with a single dose of 5e6 CAR T cells (1:1 CD8/CD4 mixture) on day 10 (MLLr B-ALL PDX) or day 3 (parental and CD19-SEM MLLr B-ALL). Tumor burden was assessed weekly for five weeks via bioluminescent imaging (BLI), then mice were followed for survival. Survival curves and tumor burden via BLI for mice that received (a) MLLr B-ALL PDX and were treated on day 10 with different CAR T cells (n=6 mice per arm); (b) SEM B-ALL cells, treated on day 3 with different CAR T cells (n=6/arm); (c) CD19-negative SEM B-ALL cells, treated on day 3 with different CAR T cells (n=6/arm). p-values were computed using the log-rank test comparing different CAR constructs to Empty CAR controls, except for 8c where CD72 CAR is compared directly to CD19 CAR.

### DETAILED DESCRIPTION OF THE DISCLOSURE

### Terminology

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein refers to the usual error range for the respective value readily known to the skilled person in this technical field. For example, for K_{D} and IC₅₀ values ±20%, ±10%, or ±5%, are within the intended meaning of the recited value.

“B-cell differentiation antigen CD72” or “CD72” (also referred to as lyb-2) is used herein to refer to a polypeptide that is encoded by a CD72 gene cytogenetically localized to human chromosome 9p13.3 (genomic coordinates (GRCh38/hg38 assembly December 2013: 9:35,609,978-35,618,426) and plays a role in B-cell proliferation and differentiation. A human CD72 protein sequence encoded by the CD72 gene is available under Uniprot accession number P21854. CD72 is a single-pass Type-II membrane protein with an extracellular C-type lectin domain and cytoplasmic ITIM motifs. CD72 has been shown to interact with the B-cell receptor complex and play a role in the normal function of B-cell signaling. It is similar to the CD22 receptor which also possesses cytoplasmic ITIM motifs. The ITIM motifs of CD72 and CD22 both function to bind to SHP-1, a protein that can interact with members of the BCR signaling chain and suppress BCR signaling as part of shaping B-cell immune tolerance. Genetic ablation of CD72 in mice was not lethal, but such mice exhibited increased immune system activation, lending evidence to its roles as a BCR inhibitory molecule. CD72 therefore is considered to be an inhibitory receptor for BCR signaling.

The term “nanobody” as used herein refers to a single-domain antibody comprising a single monomeric variable antibody domain that can form a functional antigen binding site without interaction with another variable domain, e.g., without a VH/VL interaction as is required between the VH and VL domains of a conventional 4-chain monoclonal antibody). As further detailed below, in some embodiments, a nanobody of the present invention can be incorporated into antibodies having various formats, including, e.g., a bivalent or multivalent antibody format that comprises other antibody binding domains, which may have the same, or a different, binding specificity. A nanobody of the present invention may thus be part of a larger molecule such as a multivalent or multispecific immunoglobulin that includes more than one moiety, domain or unit. A nanobody may also be part of a larger molecule that comprises another functional element, such as, for example, a half-life extender (HLE), targeting unit and/or a small molecule such a polyethyleneglycol (PEG). The term “nanobody” includes humanized versions of the nanobodies as described herein.

As used herein, “V-region” refers to an antibody, e.g., nanobody, variable region domain comprising the segments of Framework 1, CDR1, Framework 2, CDR2, and Framework 3, including CDR3 and Framework 4, which segments are added to the V-segment as a consequence of rearrangement of V-region genes during B-cell differentiation.

As used herein, “complementarity-determining region (CDR)” refers to the three hypervariable regions (HVRs) that interrupt the four “framework” regions of s variable domain. The CDRs are the primary contributors to binding to an epitope of an antigen. The CDRs of are referred to as CDR1, CDR2, and CDR3, numbered sequentially starting from the N-terminus. The term “CDR” may be used interchangeably with “HVR”.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, Md. (1991)). Chothia CDRs are determined as defined by Chothia (see, e.g., Chothia and Lesk J. Mol. Biol. 196:901-917 (1987)).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “valency” as used herein refers to the number of different binding sites of an antibody for an antigen. A monovalent antibody comprises one binding site for an antigen. A multivalent antibody comprises multiple binding sites.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this invention, the antibody binds to CD72 with a K_{D} that is at least 100-fold greater than its affinity for other antigens.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a variable domain polypeptide “corresponds to” an amino acid in the variable domain polypeptide of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

The terms “subject”, “patient” or “individual” are used herein interchangeably to refer to any mammal, including, but not limited to, a human. For example, the animal subject may be, a primate (e.g., a monkey, chimpanzee), a livestock animal (e.g., a horse, a cow, a sheep, a pig, or a goat), a companion animal (e.g., a dog, a cat), a laboratory test animal (e.g., a mouse, a rat, a guinea pig), or any other mammal. In some embodiments, the subject”, “patient” or “individual” is a human.

### Anti-CD72 Nanobodies

Provided herein are anti-CD72 nanobodies that can be used for diagnostic and therapeutic purposes.

In some embodiments, an anti-CD72 nanobody of the present disclosure has a K_{D} less than about 10 nM.

In some embodiments, an anti-CD72 nanobody of the invention has at least one, at least two, or three CDRs of a variable domain sequence of any one of SEQ ID NOS:1-8. In some embodiments, an anti-CD72 nanobody of the present invention comprises a CDR3 selected from the CDR3 sequences of a variable domain sequence of any one of SEQ ID NOS:1-8. In some embodiments, an anti-CD72 nanobody of the present invention comprises a CDR3 selected from the CDR3 sequences of a variable domain sequence of any one of SEQ ID NOS:4, 5, or 6. In some embodiments, an anti-CD72 nanobody of the present invention comprises a CDR3 of a variable domain sequence of SEQ ID NO:6. In some embodiments, an anti-CD72 nanobody of the present invention comprises a CDR1, CDR2, and CDR3 of a variable domain sequence of any one of SEQ ID NOS:1-8. In some embodiments, an anti-CD72 nanobody of the present invention comprises a CDR1, CDR2, and CDR3 of a variable domain sequence of any one of SEQ ID NOS:4, 5, or 6. In some embodiments, an anti-CD72 nanobody of the present invention comprises a CDR1, CDR2, and CDR3 of the variable domain sequence of SEQ ID NO:6.

In some embodiments, an anti-CD72 nanobody of the invention has at least one, at least two, or three CDRs of a variable domain sequence of any one of SEQ ID NOS:9-26. In some embodiments, an anti-CD72 nanobody of the present invention comprises a CDR3 selected from the CDR3 sequences of a variable domain sequence of any one of SEQ ID NOS:9-26.

In some embodiments, an anti-CD72 nanobody comprises a variable region that comprises a CDR3 of any one of SEQ ID NOS:1, 2, 5, 6, 7, or 8 in which 1, 2, 3, or 4 amino acids are substituted, e.g., conservatively substituted. In some embodiments, an anti-CD72 nanobody comprises a variable region that comprises a CDR3 of SEQ ID NO:3 in which 1, 2, or 3 amino acids are substituted, e.g., conservatively substituted. In some embodiments, an anti-CD72 nanobody comprises a CDR3 of SEQ ID NO:4 in which 1 amino acid is substituted, e.g., conservatively substituted. In some embodiments, a single chain variable region further comprises a CDR1 of any one of SEQ ID NOS:1 to 8 in which 1, 2, or 3, e.g., 1 or 2 amino acids, are substituted, e.g., conservatively substituted; and/or a CDR2 as shown in one of SEQ ID NOS:1-8 in which 1, 2, 3, or 4 amino acids are substituted, e.g., conservatively substituted. In some embodiments, an anti-CD72 nanobody comprises a variable region that comprises: a CDR1 of SEQ ID NO:6, or a variant thereof in which 1 or 2 amino acids are substituted, e.g., conservatively substituted; a CDR2 of SEQ ID NO:6; or a variant thereof in which 1, 2, or 3 amino acids are substituted, e.g., conservatively substituted; and a CDR3 of SEQ ID NO:6, or a variant thereof in which 1, 2, or 3 amino acids are substituted, e.g., conservatively substituted.

In some embodiments, an anti-CD72 nanobody comprises a variable region that comprises a CDR3 of any one of SEQ ID NOS:9-26 in which 1, 2, or 3 amino acids are substituted, e.g., conservatively substituted. In some embodiments, a single chain variable region further comprises a CDR1 of any one of SEQ ID NOS:9 to 26 in which 1, 2, or 3, e.g., 1 or 2 amino acids, are substituted, e.g., conservatively substituted; and/or a CDR2 as shown in one of SEQ ID NOS:9-26 in which 1, 2, 3, or 4 amino acids are substituted, e.g., conservatively substituted.

In some embodiments, an anti-CD72 nanobody of the present invention comprises a single chain variable region having at least 70%, 75%, 85%, 90%, 91%, 92%, 93%, 94%, 95%, 96%, 97%, 98%, 99%, or 100% sequence identity to the amino acid sequence of a variable region sequence of any one of SEQ ID NOS:1-8. In some embodiments, the variable domain comprises substitutions, insertions, or deletions in the framework of a variable region as shown in any one of SEQ ID NOS:1-8. In some embodiments, a nanobody of the present invention comprises an FR1-FR2-FR3-FR4 framework sequence that has at least 80% or at least 85% identity to the FR1-FR2-FR3-FR4 framework sequence of any one of SEQ ID NOS:1-8. In this context, FR1-FR2-FR3-FR4 is intended to refer to the framework sequence across its length, i.e., the sequence of SEQ ID NOS:1-8 from the N-terminus to the C-terminus without the three CDR sequences.

In some embodiments, an anti-CD72 nanobody of the present invention comprises a single chain variable region having at least 70%, 75%, 85%, 90%, 91%, 92%, 93%, 94%, 95%, 96%, 97%, 98%, 99%, or 100% sequence identity to the amino acid sequence of a variable region sequence of any one of SEQ ID NOS:9-26 In some embodiments, the variable domain comprises substitutions, insertions, or deletions in the framework of a variable region as shown in any one of SEQ ID NOS:9-26. In some embodiments, a nanobody of the present invention comprises an FR1-1-R2-FR3-FR4 framework sequence that has at least 80% or at least 85% identity to the FR1-FR2-FR3-FR4 framework sequence of any one of SEQ ID NOS:9-26. In this context, FR1-FR2-FR3-FR4 is intended to refer to the framework sequence across its length, i.e., the sequence of SEQ ID NOS:9-26 from the N-terminus to the C-terminus without the three CDR sequences.

In some embodiments, the FR1 region of a nanobody of the present invention comprises an FR1 sequence having at least 80%, at least 85%, at least 90%, or at least 95% identity to the FR1 sequence of any one of SEQ ID NOS:1-8. In some embodiments, the FR1 region of a nanobody of the present invention comprises an FR1 sequence having at least 80%, at least 85%, at least 90%, or at least 95% identity to the FR1 sequence of any one of SEQ ID NOS:9-26.

In some embodiments, the FR2 region of a nanobody of the present invention comprises an FR2 sequence having at least 80%, at least 85%, at least 95%, at least 90%, or at least 95% identity to the FR2 sequence of any one of SEQ ID NOS:1-8. In some embodiments, the FR2 region of a nanobody of the present invention comprises an FR2 sequence having at least 80%, at least 85%, at least 90%, or at least 95% identity to the FR2 sequence of any one of SEQ ID NOS:9-26.

In some embodiments, the FR3 region of a nanobody of the present invention comprises an FR3 sequence having at least 80%, at least 85%, at least 90%, or at least 95% identity to the FR3 sequence of any one of SEQ ID NOS:1-8. In some embodiments, the FR3 region of a nanobody of the present invention comprises an FR3 sequence having at least 80%, at least 85%, at least 90%, or at least 95% identity to the FR3 sequence of any one of SEQ ID NOS:9-26.

In some embodiments, the FR4 region of a nanobody of the present invention comprises an FR4 sequence having at least 80%, at least 85%, at least 90%, or at least 95% identity to the FR4 sequence of any one of SEQ ID NOS:1-8. In some embodiments, the FR4 region of a nanobody of the present invention comprises an FR4 sequence having at least 80%, at least 85%, at least 90%, or at least 95% identity to the FR4 sequence of any one of SEQ ID NOS:9-26.

As previously explained, a nanobody of the present invention may be incorporated into a bivalent antibody or a multivalent antibody that binds to the same, or a different, antigen. In some embodiments, a nanobody of the present invention may be incorporated into a bispecific antibody or multispecific antibody that binds to the an antigen at different epitopes, or that binds to different antigens. In some embodiments, such an antibody may comprise an Fc region. In some embodiments, a nanobody of the present invention may be present as an antigen binding domain of a larger molecule, e.g., present as an antigen binding domain of a chimeric antigen receptor or synthetic Notch receptor, as further detailed below. In further embodiments, a bispecific antibody, multispecific antibody, chimeric antibody receptor, synthetic Notch receptor, or other nanobody-containing construct, may comprises more than one anti-CD72 nanobody as described herein, e.g., two, three, or four anti-CD72 nanobodies of the present invention, e.g., where the nanobodies are joined by linkers.

In some embodiments, a nanobody of the present invention is linked to a second nanobody, e.g., a second anti-CD72 nanobody as described herein, or to an scFV antibody to form a bi-specific antibody. Thus, for example, in some aspects, an anti-CD72 nanobody of the present invention may be incorporated into a bispecific antibody having a second binding domain that targets an antigen on an immune effector cell, such as a T cell. Accordingly, in some embodiments, a bispecific antibody may comprise an anti-CD72 nanobody of the present invention and an antibody, e.g., scFv, that targets CD3 or an anti-CD16 scFv for engaging NK cells. In some embodiments, a bispecific antibody comprises an anti-CD72 nanobody as described herein and an antibody, e.g., scFV, that targets CD28.

### CAR Constructs Comprising an Anti-CD72 Nanobody

Chimeric antigen receptors (CARs) are recombinant receptor constructs comprising an extracellular antigen-binding domain (e.g., a nanobody) joined to a transmembrane domain, and further linked to an intracellular signaling domain (e.g., an intracellular T cell signaling domain of a T cell receptor) that transduces a signal to elicit a function. In certain embodiments, immune cells (e.g., T cells or natural killer (NK) cells) are genetically modified to express CARs that comprise one or more anti-CD72 nanobodies of the present and have the functionality of effector cells (e.g., cytotoxic and/or memory functions of T cells or NK cells).

In a standard CAR, the components include an extracellular targeting domain, a transmembrane domain and intracellular signaling/activation domain, which are typically linearly constructed as a single fusion protein. In the present invention, the extracellular region comprises an anti-CD72 nanobody as described herein. The “transmembrane domain” is the portion of the CAR that links the extracellular binding portion and intracellular signaling domain and anchors the CAR to the plasma membrane of the host cell that is modified to express the CAR, e.g., the plasma membrane of an immune effector cell. The intracellular region may contain a signaling domain of TCR complex, and/or one or more costimulatory signaling domains, such as those from CD28, 4-1BB (CD137) and OX-40 (CD134). For example, a “first-generation CAR” generally has a CD3-zeta signaling domain. Additional costimulatory intracellular domains may also be introduced (e.g., second and third generation CARS) and further domains including homing and suicide domains may be included in CAR constructs. CAR components are further described below.

### Extracellular Domain (Nanobody Domain)

A chimeric antigen receptor of the present disclosure comprises an extracellular antigen-binding domain that comprises an anti-CD72 nanobody domain having a CDR1, CDR2, and CDR3 as described herein. In some embodiments, the anti-CD72 nanobody domain comprises a humanized version of any one of SEQ ID NOS:1-8, e.g., in which residues in the framework are substituted to provide a framework sequence FR1-FR2-FR3-FR4 that has at least 85%, or at least 90%, or at least 95%, or greater, to a human VH framework, e.g., a human germline framework, FR1-FR2-FR3-FR4. In some embodiments, the anti-CD72 nanobody domain comprises a humanized version of any one of SEQ ID NOS:9-26, e.g., in which residues in the framework are substituted to provide a framework sequence FR1-FR2-FR3-FR4 that has at least 85%, or at least 90%, or at least 95%, or greater, to a human VH framework e.g., a human germline framework, FR1-FR2-FR3-FR4.

In some embodiments, the extracellular domain may comprise two more anti-CD72 nanobodies as described herein. For example, the extracellular domain may comprise three of four different nanobodies that are described herein. In some embodiments, the extracellular domain may comprises multiple copies of the same nanobody. In some embodiments, the extracellular domain may comprise a nanobody as described herein and an anti-CD72 nanobody, or other anti-CD72 antibody, that binds to a different CD72 epitope. In some embodiments at least one of the nanobodies comprises a CDR1 sequence comprising TISPIDI, a CDR2 sequence comprising FVAAIALGGN, and a CDR3 sequence comprising VGYVDKWDDSDYHT.

A CAR construct encoding a CAR may also comprise a sequence that encodes a signal peptide to target the extracellular domain to the cell surface.

### Hinge Domain

In some embodiments, the CAR may one or more hinge domains that link the antigen binding domain comprising an anti-CD72 nanobody of the present invention and the transmembrane domain for positioning the antigen binding domain. Such a hinge domain may be derived either from a natural, synthetic, semi-synthetic, or recombinant source. The hinge domain can include the amino acid sequence of a naturally occurring immunoglobulin hinge region, e.g., a naturally occurring human immunoglobulin hinge region, or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered.

### Transmembrane Domain

Any transmembrane suitable for use in a CAR construct may be employed. Such transmembrane domains, include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD 18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100, (SEMA4D), SLAMF6 (NTB-A, Ly108), SLAM (SLAMF1, CD150, IPO-3), BLAME, (SLAMF8), SELPLG (CD162), LTBR, PAG/Cbp, NKG2D, or NKG2C.

A transmembrane domain incorporated into a CAR construct may be derived either from a natural, synthetic, semi-synthetic, or recombinant source.

### Intracellullar Signaling Domain

A CAR construct of the present disclosure includes one or more intracellular signaling domains, also referred to herein as co-stimulatory domains, or cytoplasmic domains that activate or otherwise modulate an immune cell, (e.g., a T lymphocyte or NK cell). The intracellular signaling domain is generally responsible for activation of at least one of the normal effector functions of the immune cell in which the CAR has been introduced. In one embodiment, a co-stimulatory domain is used that increases CAR immune T cell cytokine production. In another embodiment, a co-stimulatory domain is used that facilitates immune cell (e.g., T cell) replication. In still another embodiment, a co-stimulatory domain is used that prevents CAR immune cell (e.g., T cell) exhaustion. In another embodiment, a co-stimulatory domain is used that increases immune cell (e.g., T cell) antitumor activity. In still a further embodiment, a co-stimulatory domain is used that enhances survival of CAR immune cells (e.g., T cells) (e.g., post-infusion into patients).

Examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of IT AM containing primary intracellular signaling domains include those of CD3 zeta, common FcR gamma, Fc gamma R11a, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

An intracellular signaling domain of a CAR can comprise a primary intracellular signaling domain only, or may comprise additional desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM, (SLAMF1, CD150, IPO-3), BLAME (SLAMF8), SELPLG (CD162), LTBR, LAT, GADS, SLP-76, PAG/Cbp, and CD 19a.

In some embodiments, a CAR may be designed as an inducible CAR, or may otherwise comprise a mechanisms for reversibly expressing the CAR, or controlling CAR activity to largely restrict it to a desired environment. Thus, for example, in some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

In some embodiments, a host cell, e.g., a T cell, can be engineered such that a synthetic Notch receptor comprising an extracellular domain that targets one antigen induces the expression of a CAR that targets a second antigen. Such systems are described, e.g., in U.S. Patent Application Publication No. 20190134093; see also, synNotch polypeptides as described in US20160264665, each incorporated herein by reference. In some embodiments, a synNotch comprises a one or more anti-CD72 nanobodies as described herein. In some embodiments, one or more anti-CD72 nanobodies is incorporated into a CAR, the expression of which is activated by a synNotch expressed by the host cell.

In some embodiments, a cell expressing a CAR comprising one or more anti-CD72 nanobodies as described herein also expresses a second CAR, e.g., a second CAR that includes a different antigen binding domain, e.g., that binds to the same target or a different target (e.g., a target other than CD72, e.g., CD22 or CD19, that is expressed on a B cell malignancy.

### Activation and Expansion of Immune Effector Cells (e.g., T Cells)

The invention is not limited by the type of immune cells genetically modified to express a CAR, or synthetic Notch receptor. Illustrative immune cells include, but are not limited to, T cells, e.g., alpha/beta T cells and gamma/delta T cells, B cells, natural killer (NK) cells, natural killer T (NKT) cells, mast cells, macrophages, and myeloid-derived phagocytes. T cells that can be modified to express CARs include memory T cells, CD4+, and CD8+ T cells. In some embodiments, the immune cells, e.g., T cells, are autologous cells from the patient to undergo immunotherapy. In some embodiments, the immune cells are allogeneic.

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Examples of immune effector cells include T cells, e.g., alpha/beta T cells and gamma/delta T cells, B cells, natural killer (NK) cells, natural killer T (NKT) cells, mast cells, and myeloid-derived phagocytes.

Methods of making CAR-expressing cells are described, e.g., in US2016/0185861 and US2019/0000880.

### Nucleic Acids and Vectors Encoding CARS

Any method may be used to genetically modify an effector cells, such as a T-cell or NK cell to express a CAR comprising an anti-CD72 nanobody of the present invention. Non-limiting examples of methods of genetically engineering immune cells include, but are not limited to, retrovirus- or lentivirus-mediated transduction. Other viral delivery systems include adenovirus, adeno-associated virus, herpes simplex viral vectors, pox viral vectors, alphavirus vectors, poliovirus vectors, and other positive and negative stranded RNA viruses, viroids, and virusoids, or portions thereof. Methods of transduction include direct co-culture of the cells with producer cells, e.g., by the method of Bregni, et al. Blood 80: 1418-1422 (1992), or culturing with viral supernatant alone or concentrated vector stocks with or without appropriate growth factors and polycations, e.g., by the method of Xu, et al. Exp. Hemat. 22:223-230 (1994); and Hughes, et al. J. Clin. Invest. 89: 1817 (1992).

In some embodiments, genetic modification is performed using transposase-based systems for gene integration, CRISPR/Cas-mediated gene integration, TALENS or Zinc-finger nucleases integration techniques. For example, CRISPR/Cas-mediated gene integration may be employed to introduce a CAR or synthetic Notch receptor into immune effectors cells, which may then be selected and expanded for administration to a patient.

### Nanobody Conjugates

In a further aspect, an anti-CD72 nanbody of the present invention may be conjugated or linked, either directly or indirectly, to therapeutic and/or imaging/detectable moieties. For example, in some embodiments, a nanobody or the present invention, or an antigen binding region comprising a nanobody of the present invention, may be conjugated to agents including, but not limited to, a detectable marker, a cytotoxic agent, an imaging agent, a therapeutic agent, or an oligonucleotide. Methods for conjugating or linking a nanobody, or antigen binding regions comprising a nanobody, to a desired molecule moiety are well known in the art. The moiety may be linked to the nanobody covalently or by non-covalent linkages.

In some embodiments, an anti-CD72 nanobody of the present invention, or an antigen binding domain comprising an anti-CD72 nanobody of the present invention, is conjugated to cytotoxic moiety or other moiety that inhibits cell proliferation. In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracin dione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis.

In some embodiments, an anti-CD72 nanobody of the present invention, or an antigen binding domain comprising an anti-CD72 nanobody of the present invention, may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### Cancer Vaccines

An anti-CD72 nanobody, an antigen binding molecule comprising an anti-CD72 nanobody, or an effector cell, e.g., T-cell, genetically modified a CAR comprising an anti-CD72 nanobody of the present invention can be combined with an immunogenic agent, such as cancerous cells, purified tumor antigens (including recombinant proteins, peptides, and carbohydrate molecules), and cells transfected with genes encoding immune stimulating cytokines (He et al. (2004) J. Immunol. 173:4919-28). Non-limiting examples of cancer vaccines that can be used include t cells transfected to express the cytokine GM-CSF, DNA-based vaccines, RNA-based vaccines, and viral transduction-based vaccines. The cancer vaccine may be prophylactic or therapeutic.

In some embodiments, an anti-CD72 nanobody, an antigen binding molecule comprising an anti-CD72 nanobody, or an effector cell, e.g., T-cell, genetically modified a CAR comprising an anti-CD72 nanobody of the present invention is co-administered with an immunomodulating agent. Examples of immodulating agents include, but are not limited to, cytokines, growth factors, lymphotoxins, tumor necrosis factor (TNF), hematopoietic factors, interleukins (e.g., interleukin-1 (IL-1), IL-2, IL-3, IL-6, IL-10, IL-12, IL-15, an IL-15/IL-15Rα, e.g., sushi domain, complex, IL-18, and IL-21), colony stimulating factors (e.g., granulocyte-colony stimulating factor (G-CSF) and granulocyte macrophage-colony stimulating factor (GM-CSF), interferons (e.g., interferon-α, -β or -γ), erythropoietin and thrombopoietin, or a combination thereof. In some embodiments, the complex may be co-administered with an adjuvant, such as a Toll-like receptor (TLR) agonist, a C-type lectin receptor (CLR) agonist, a retinoic acid-inducible gene I-like receptor (RLR) agonist, a saponin, a polysaccharide such as chitin, chitosan, β-glucan, an ISCOM, QS-21, or another immunopotentiating agent.

### Treatment of B-Cell Malignancies

An anti-CD72 nanobody of the present invention, including embodiments in which the anti-CD72 nanobody is provided as a component of an antigen binding molecule, such as a bivalent or multivalent antibody, or is provided as a component of a CAR molecule, can be used to treat any malignancy that expresses CD72. In some embodiments, the malignancy is a B cell malignancy. Illustrative B-cell malignancies include, but are not limited to, B-cell acute lymphoblastic leukemia, chronic lymphocytic leukemia/small lymphocytic lymphoma, monoclonal B-cell lymphocytosis, B-cell prolymphocytic leukemia, splenic marginal zone lymphoma, hairy cell leukemia, splenic B-cell lymphoma/leukemia, unclassifiable, splenic diffuse red pulp small B-cell lymphoma, hairy cell leukemia-variant, lymphoplasmacytic lymphoma, Waldenstrom macroglobulinemia, monoclonal gammopathy of undetermined significance (MGUS) IgM, μ, heavy-chain disease, γ heavy-chain disease, a heavy-chain disease, MGUS IgG/A, plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, In situ follicular neoplasia, duodenal-type follicular lymphoma, pediatric-type follicular lymphoma, large B-cell lymphoma with IRF4 rearrangement, primary cutaneous follicle center lymphoma, mantle cell lymphoma, in situ mantle cell neoplasia, diffuse large B-cell lymphoma (DLBCL) NOS, including germinal center B-cell type, and activated B-cell type; T-cell/histiocyte-rich large B-cell lymphoma, primary DLBCL of the central nervous system, primary cutaneous DLBCL, leg type, EBV DLBCL NOS, EBV mucocutaneous ulcer, DLBCL associated with chronic inflammation, lymphomatoid granulomatosis, primary mediastinal (thymic) large B-cell lymphoma, intravascular large B-cell lymphoma, ALK⁺ large B-cell lymphoma, plasmablastic lymphoma, primary effusion lymphoma, HHV8⁺ DLBCL NOS, Burkitt lymphoma, Burkitt-like lymphoma with 11q aberration, high-grade B-cell lymphoma, with MYC and BCL2 and/or BCL6 rearrangements, high-grade B-cell lymphoma NOS, and B-cell lymphoma, unclassifiable, with features intermediate between DLBCL and classical Hodgkin lymphoma. In some embodiments, a malignancy treated with an anti-CD72 nanobody as described herein is Hodgkin lymphoma, e.g., nodular lymphocyte predominant Hodgkin lymphoma, or classical Hodgkin lymphoma, including nodular sclerosis classical Hodgkin lymphoma, lymphocyte-rich classical Hodgkin lymphoma, mixed cellularity classical Hodgkin lymphoma, and lymphocyte-depleted classical Hodgkin lymphoma. In some embodiments a malignancy treated with an anti-CD72 nanobody as described herein in a posttransplant lymphoproliferative disorder (PTLD), such as plasmacytic hyperplasia PTLD, infectious mononucleosis PTLD, florid follicular hyperplasia PTLD, polymorphic PTLD, monomorphic PTLD (B- and T-/NK-cell types), or classical Hodgkin lymphoma PTLD.

### Administration of Anti-CD72 Nanobody

In one aspect, a method of treating a B-cell malignancy using an anti-CD72 nanobody or antigen binding molecule, e.g., an antibody, that comprises the anti-CD72 nanobody comprises administering the anti-CD72 nanobody or antigen binding molecule that comprises the anti-CD72 nanobody, as a pharmaceutical composition to a patient in a therapeutically effective amount using a dosing regimen suitable for treatment of the B-cell malignancy. The composition can be formulated for use in a variety of drug delivery systems. One or more physiologically acceptable excipients or carriers can also be included in the compositions for proper formulation. Suitable formulations for use in the present invention are found, e.g., in Remington: The Science and Practice of Pharmacy, 21st Edition, Philadelphia, Pa. Lippincott Williams & Wilkins, 2005.

The nanobody (or antibody or antigen binding molecule comprising the nanobody) is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include the amelioration of symptoms of the disease in the patient. Amounts effective for this use will depend upon the severity of the disease and the general state of the patient's health, including other factors such as age, weight, gender, administration route, etc. Single or multiple administrations of the antibody may be administered depending on the dosage and frequency as required and tolerated by the patient. In any event, the methods provide a sufficient quantity of anti-CD72 nanobody or antigen binding molecule that comprises the anti-CD72 nanobody to effectively treat the patient.

The nanobody can be administered by any suitable means, including, for example, parenteral, intrapulmonary, and intranasal administration. Parenteral infusions include intramuscular, intravenous, intraarterial, intraperitoneal, or subcutaneous administration. In some embodiments, the nanobody may be administered by insufflation. In an illustrative embodiment, the nanobody may be stored at 10 mg/ml in sterile isotonic aqueous saline solution for injection at 4° C. and is diluted in either 100 ml or 200 ml 0.9% sodium chloride for injection prior to administration to the patient. In some embodiments, the nanobody is administered by intravenous infusion over the course of 1 hour at a dose of between 0.01 and 25 mg/kg. In other embodiments, the nanobody is administered by intravenous infusion over a period of between 15 minutes and 2 hours. In still other embodiments, the administration procedure is via sub-cutaneous bolus injection.

The dose of nanobody is chosen in order to provide effective therapy for the patient and is in the range of less than 0.01 mg/kg body weight to about 25 mg/kg body weight or in the range 1 mg-2 g per patient. Preferably the dose is in the range 0.1-10 mg/kg or approximately 50 mg-1000 mg/patient. The dose may be repeated at an appropriate frequency which may be in the range once per day to once every three months, or every six months, depending on the pharmacokinetics of the nanobody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the nanobody is administered approximately once per month.

### Administration of Immune Effector Cells Comprising an Anti-CD72 Nanobody

In some embodiments, pharmaceutical compositions of the present invention comprise a CAR-expressing immune effector cells e.g., a plurality of CAR-expressing immune effector cells that are genetically modified to express a CAR comprising an anti-CD72 nanobody as described herein. Such cells may be formulated with one or more pharmaceutically or physiologically acceptable carriers, diluents or excipients, e.g., buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, immune effector cells genetically modified to express a CAR comprising an anti-CD72 nanobody are formulated for intravenous administration.

Pharmaceutical compositions comprising the CAR-modified immune effector cells may be administered in a manner appropriate to the B-cell malignancy to be treated. The quantity and frequency of administration will be determined by such factors as the condition of the patient, and the type and severity of the patient's disease, although appropriate dosages may be determined by clinical trials.

In some embodiments, a pharmaceutical composition comprising CAR-modified immune effector cells, e.g., T cells or NK cells, as described herein are administered at a dosage of 10⁴ to 10⁹ cells/kg body weight, in some instances 10⁵ to 10⁶ cells/kg body weight, including all integer values within those ranges. In some embodiments, the cells, e.g., T cells or NK cells modified as described herein, may be administered at 3×10⁴, 1×10⁶, 3×10⁶, or 1×10⁷ cells/kg body weight. The cell compositions may also be administered multiple times at these dosages. Administration can be performed using infusion techniques that are commonly known in immunotherapy (see, e.g., Rosenberg et al., New Eng. J. of Med. 319: 1676, 1988). In some embodiments, the genetically modified immune effector cells are administered intravenously. In such cells are administered to a patient by intradermal or subcutaneous injection. The CAR-expressing cells may also be injected directly in to a particular site, such as a lymph node.

In some embodiments, a, subject may undergo leukapheresis, wherein leukocytes are collected, enriched, or depleted ex vivo to select and/or isolate the cells of interest, e.g., T or NK cells. These cell isolates, e.g., T cell or NK cell isolates, may be expanded by methods known in the art and treated such that one or more CAR constructs of the invention may be introduced, thereby creating a CAR-expressing cell, e.g., CAR-T cell or CAR-expressing NK cell, of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells of the present invention. In an additional aspect, expanded cells are administered before or following surgery.

In embodiments, lymphodepletion, e.g., using melphalan, cytoxan, cyclophosphamide, or fludarabind, is performed on a subject, e.g., prior to administering a population of immune effectors cells that express a CAR comprising an anti-CD72 nanobody of the present invention.

In one embodiment, a CAR is introduced into cells, e.g., T cells or NK cells, e.g., using in vitro transcription, and the subject (e.g., human) receives an initial administration of CAR-expressing cells, e.g., CAR T cells or CAR-expressing NK cells of the invention, and one or more subsequent administrations of the CAR-expressing cells, e.g., CAR T cells or CAR-expressing NK cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells, e.g., CAR T cells or CAR-expressing NK cells of the invention are administered to the subject (e.g., human) per week, e.g., 2, 3, or 4 administrations of the CAR-expressing cells, e.g., CAR T cells or CAR-expressing NK cells of the invention are administered per week. In one embodiment, the subject (e.g., human subject) receives more than one administration of the CAR-expressing cells, e.g., CAR T cells per week or CAR-expressing NK cells (e.g., 2, 3 or 4 administrations per week) (also referred to herein as a cycle), followed by a week of no CAR-expressing cells, e.g., CAR T cell administrations or CAR-expressing NK cell administrations, and then one or more additional administration of the CAR-expressing cells, e.g., CAR T cells or CAR-expressing NK cells (e.g., more than one administration of the CAR-expressing cells, e.g., CAR T cells or CAR-expressing NK cells, per week) is administered to the subject. In another embodiment, the subject (e.g., human subject) receives more than one cycle of CAR-expressing cells, e.g., CAR T cells or CAR-expressing NK cells, and the time between each cycle is less than 10, 9, 8, 7, 6, 5, 4, or 3 days. In one embodiment, the CAR-expressing cells, e.g., CAR-T cells or CAR-expressing NK cells, are administered every other day for 3 administrations per week. In one embodiment, the CAR-expressing cells, e.g., CAR T cells or CAR-expressing NK cells of the invention, are administered for at least two, three, four, five, six, seven, eight or more weeks.

In some embodiments, CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97

Administration in Combination with Other Agents

An anti-CD72 nanobody of the present disclosure (or antibody or antigen binding molecule comprising the nanobody), or immune effector cells genetically modified to express a nanobody as described herein may be administered with one or more additional therapeutic agents, e.g., radiation therapy, chemotherapeutic agents and/or immunotherapeutic agents. As used herein, administered “in combination”, means that two (or more) different treatments are delivered to the subject for the treatment of the B-cell malignancy, e.g., the two or more treatments are administered after the subject has been diagnosed with the B-cell malignancy. In some embodiments, there may be overlap in the time frames in which the two therapeutic agents are administered. In other embodiments, one treatment protocol ends before the second begins. In some embodiment, treatment may be more effective because of combined administration.

In some embodiments, the nanobody or immune effector cells that express a CAR comprising the nanobody, are administered in conjunction with an agent that targets an immune checkpoint antigen. In one aspect, the agent is a biologic therapeutic or a small molecule. In another aspect, the agent is a monoclonal antibody, a humanized antibody, a human antibody, a fusion protein or a combination thereof. In certain embodiments, the agents inhibit, e.g., by blocking ligand binding to receptor, a checkpoint antigen that may be PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137 (4-1BB), CD160, CD39, VISTA, TIGIT, a SIGLEC, CGEN-15049, 2B4, CHK 1, CHK2, A2aR, B-7 family ligands or a combination thereof. In some embodiments, the agent targets PD-1, e.g., an antibody that blocks PD-L1 binding to PD-1 or otherwise inhibits PD-1. In some embodiments, agent targets CTLA-4. In some embodiments, the targets LAG3. In some embodiments, the agents targets TIM3. In some embodiments, the agents target ICOS.

In some embodiments, the anti-CD72 nanobody or immune effector cells expressing a CAR comprising the nanobody can be administered in conjunction with an additional therapeutic antibody that targets an antigen on a B-cell malignancy. Examples of therapeutic antibodies for the treatment of B-cell malignancies include antibodies that target CD20, CD22, and CD19, including, e.g., rituximab, obinutuzumab, tositumomab ofatumumab, veltuzumab, and ocrelizumab, epratuzumab, and blinatomomab.

In some embodiments, the anti-CD72 nanobody or immune effector cells comprising the antibody are administered with a chemotherapeutic agent. Examples of cancer chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as bexarotene, alitretinoin; denileukin diftitox; esperamicins; capecitabine; and pharmaceutically acceptable salts, acids or derivatives of any of the above. In some embodiments, anti-CD72 nanobody or immune effector cells expressing a CAR comprising the nanobody can be administered in conjunction with an additional therapeutic compound that modulates the B-cell receptor signaling complex or other members of its signaling pathway. Such compounds include agonists or antagonists of Protein Kinase C, PI3K, BTK, BLNK, PLC-gamma, PTEN, SHIP1, SHP1, SHP2, ERK, and others. Examples of therapeutic compounds that target B-cell receptor signaling and/or other members of its signaling pathway include Bryostatin 1, 3AC, RMC-4550, and SHP099.

The following examples illustrate certain aspects of the claimed invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Identification of CD72 as a Therapeutic Target for B-Cell Malignancies

### Cell Surface Proteomics of MLLr Versus Other B-ALL Subtypes Shows Distinct Surfaceome Signatures

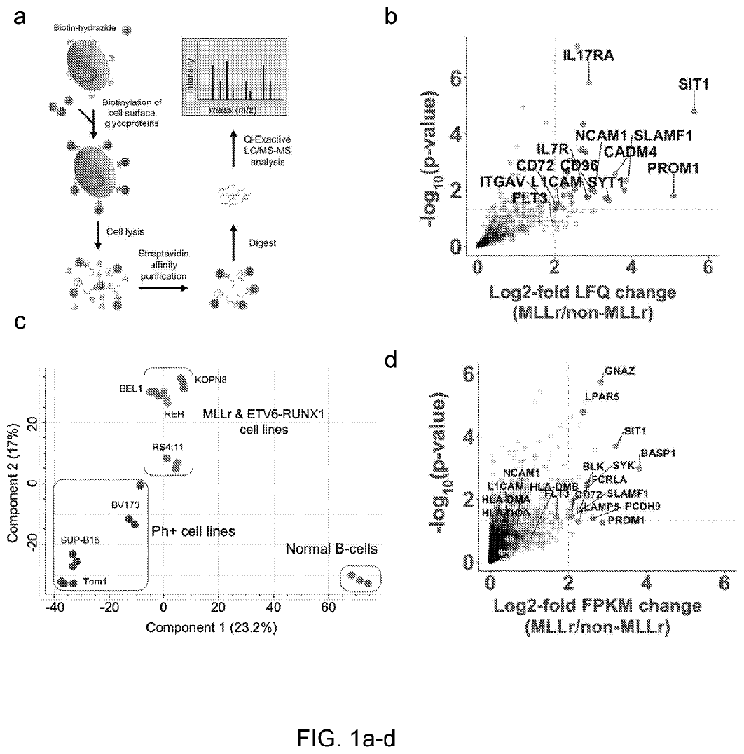

To define the B-ALL cell surfaceome, we enriched N-glycoproteins using a modified version of the Cell Surface Capture method (FIG. 1a) followed by quantitative mass spectrometry. As this method requires sample input of 30-200e6 cells, it is not routinely amenable to primary sample analysis; we therefore performed our analyses on cell lines. We profiled eight B-ALL lines with distinct driver translocations including MLL-AF4 (n=3), MLL-ENL (n=1), BCR-ABL (n=3), and ETV6-RUNX1 (n=1), plus Epstein Barr Virus-immortalized B-cells derived from normal donor umbilical cord blood as a non-malignant comparator (Table 1), all performed in biological triplicate. Using label-free quantification (LFQ) in MaxQuant and filtering for Uniprot-annotated membrane or membrane-associated proteins, we quantified 799 membrane proteins. Using a 2-fold cutoff and p<0.05 derived from a Welch's T-test between MLLr vs non-MLLr cell lines, we identified 25 unique membrane proteins specifically enriched on the MLLr surfaceome with 39 downregulated (FIG. 1b). As positive controls, our analysis identified known hallmarks of MLLr including PROM1 and FLT3 upregulation as well as loss of CD10. Principal component analysis showed marked separation of MLLr B-ALL lines from BCR-ABL B-ALL and EBV-immortalized B-cells, implying a distinct cell surfaceome (FIG. 1c). To investigate surface protein regulation, we performed parallel RNA-seq. We found a modest correlation between upregulated surface-annotated proteins found by both RNA-seq and surface proteomics, consistent with prior studies (FIG. 1d).

TABLE 1
Cell lines profiled by cell surface proteomics. Proteomic Summary:
3 biological replicates each; 3.5 × 10⁷ cell per replicate;
1,276 membrane proteins identified and quantified.

| | | Genetic | |
|---|---|---|---|
| Cell Line | Subtype | Rearrangement | Fusion Gene |
| RS4:11 | B-lineage ALL | t(4; 11)(q21; q23) | MLL-AF4 |
| SEM | B-lineage ALL | t(4; 11)(q21; q23) | MLL-AF4 |
| BEL1 | B-lineage ALL | t(4; 11)(q21; q23) | MLL-AF4 |
| KOPN-8 | B-lineage ALL | t(11; 19)(q23; p13) | MLL-ENL |
| REH | B-lineage ALL | t(12; 21)(q13; q22) | ETV6-RUNX1 |
| SUP-B15 | B-lineage ALL | t(9; 22)(q34; q11) | BCR-ABL1 |
| BV173 | B-lineage ALL | t(9; 22)(q34; q11) | BCR-ABL1 |
| TOM-1 | B-lineage ALL | t(9; 22)(q34; q11) | BCR-ABL1 |
| B-cells | Non-malignant | N/A | N/A |
| | immortalized | | |

### Triage of Cell Surface Proteins Identifies CD72 as an Immunotherapy Target

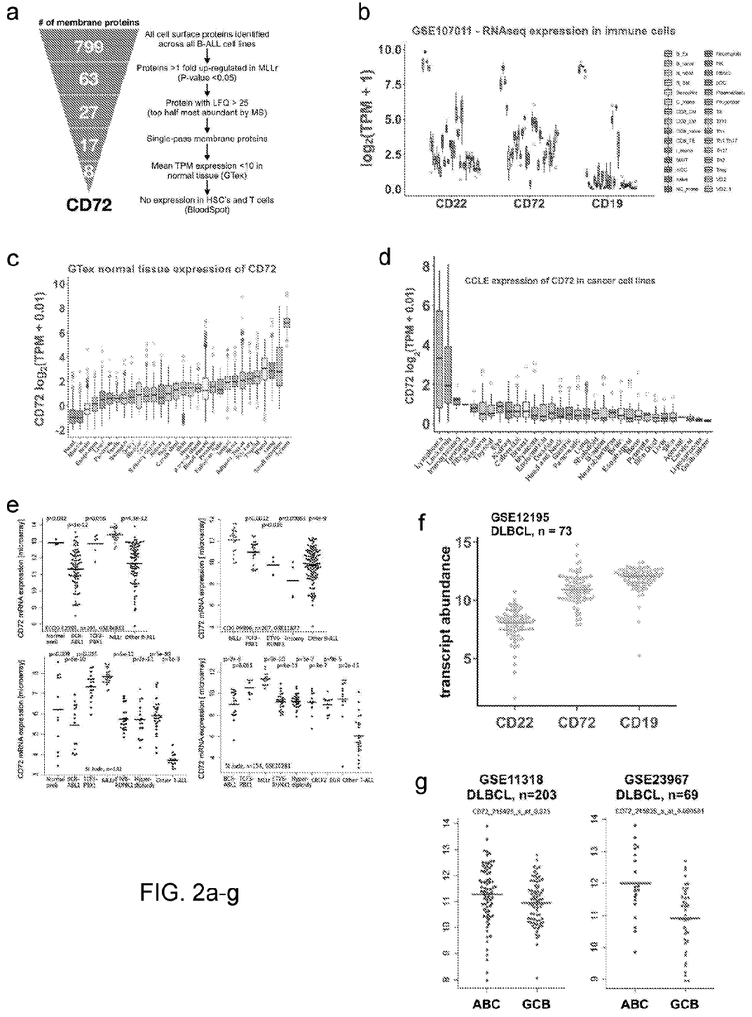

We bioinformatically triaged our cell surface markers in order to identify potential immunotherapy candidates for MLLr B-ALL. We sequentially considered up-regulated cell surface markers in MLLr vs. other (63/799 proteins); relatively abundant proteins to find markers with high antigen density (LFQ intensity >25, 27/799); and single pass membrane proteins to facilitate development of in vitro antibodies (17/799) (FIG. 2a). To avoid “on-target, off-tumor” toxicity, we eliminated protein-encoding genes with a median TPM (transcript per million) >10 in normal tissue (excluding Spleen) per Genotype-Tissue Expression database (GTex), or any detectable immunohistochemical staining in non-hematopoietic tissues per the Human Protein Atlas. This left 8 of 799 proteins. Finally, to avoid sensitive hematopoietic compartments, we eliminated proteins with any detectable RNA expression in CD34+ stem and progenitor cells (HSPCs) or T-cells in the DMAP resource and Human Blood Atlas (HBA). After completing this triage, one membrane protein target stood out as best-fulfilling our criteria: CD72.

CD72, also known as lyb-2 in murine biology, is a single-pass Type-II membrane protein with an extracellular C-type lectin domain and cytoplasmic ITIM motifs. The ITIM motifs on CD72, similar to CD22, serve as scaffolds for inhibitory phosphatases to counteract B-cell receptor (BCR) signaling. These proteins, as well as CD19, demonstrate highly similar expression patterns across hematopoietic cell types per the HBA (FIG. 2b), including low expression on most normal tissue (FIG. 2c). Investigation of CD72 transcript abundance in malignant cell lines revealed high expression in leukemia as well as lymphoma cell lines with little to no expression in malignancies originating in other tissues (FIG. 2d, n=1461; CCLE, accessed Oct. 14, 2019). Reanalysis of multiple cell line and patient sample transcriptome datasets also confirmed that CD72 is highly expressed in most B-ALL subtypes as well as the poor-prognosis subtype Diffuse Large B-Cell Lymphoma (DLBCL) (FIG. 2e-g).

### CD72 is Highly-Abundant in MLLr Leukemia as Well as Other B-Cell Malignancies

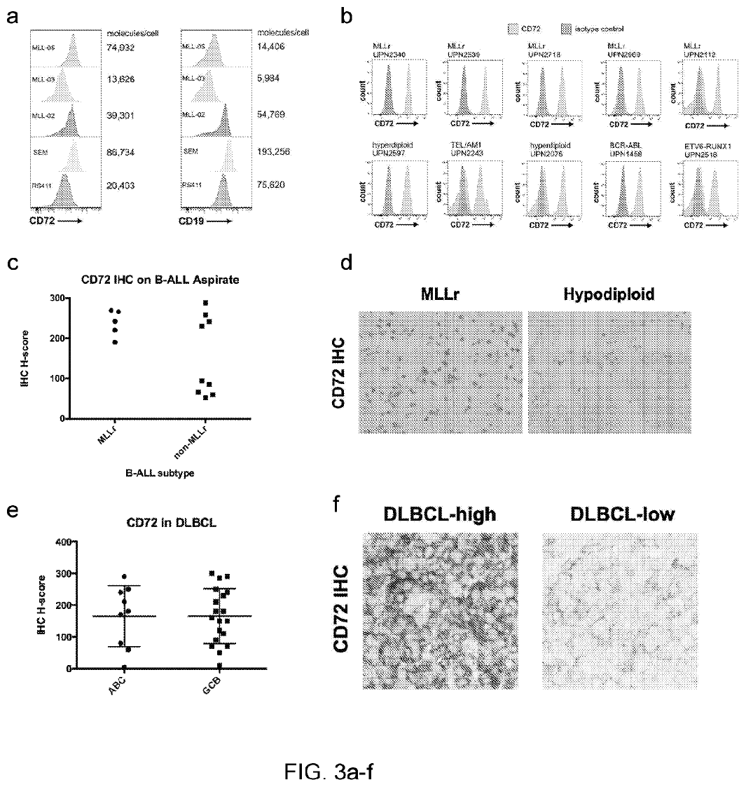

To independently verify these results, we examined CD72 surface expression on B-ALL patient-derived xenografts (PDX) from the ProXe biobank and viably frozen primary pediatric samples from our institution. By quantitative flow cytometry, we found CD72 to be expressed at several thousand copies per cell in MLLr PDX samples, similar to and sometimes greater than CD19 (FIG. 3a). Primary sample analysis suggested higher CD72 in MLLr cells than non-MLLr, but, importantly, revealed CD72 expression even in non-MLLr disease (FIG. 3b). IHC on fixed adult B-ALL bone marrow aspirate found uniformly high CD72 on MLLr B-ALL blasts, compared to variable, but still present, expression in other genomic subtypes (FIG. 3c-d). IHC was also performed examining CD72 in both activated B-cell (ABC) and germinal center B-cell (GBC) DLBCL and found that although there was no significant difference between the two subtypes, the vast majority of samples examined possessed high levels of CD72 (FIG. 3e-f). To further examine CD72 surface expression on additional lymphoma subtypes, cell surface abundance of both CD19 and CD72 were assess on human leukemia cell lines ((SEM and RS411) and human lymphoma cell lines (JEKO-1, HBL1, Namalwa, Toledo, OCI-Ly10) by quantitative flow cytometry using FITC Quantum MESF (Molecules of Equivalent Soluble Fluorochrome) beads (Bangs Laboratories) and FITC-labeled anti-CD19 and anti-CD72 monoclonal antibodies (BD). CD72 was found to be in high abundance on all cell lines examined (Table 2).

TABLE 2
Expression of CD19 and CD72 receptors on leukemia and lymphoma
cell lines Cell surface abundance of CD19 and CD72 were
measured on human leukemia cell lines (SEM and RS411) and
human lymphoma cell lines (JEKO-1, HBL1, Namalwa, Toledo,
OCI-Ly10) by quantitative flow cytometry using FITC Quantum
MESF (Molecules of Equivalent Soluble Fluorochrome) beads
(Bangs Laboratories) and FITC-labeled anti-CD19 and anti-
CD72 monoclonal antibodies (BD Biosciences)

| Cell Line | Type | Receptor | Receptor Number per Cell |
|---|---|---|---|
| SEM | Leukemia | CD19 | 219,724 |
| RS411 | Leukemia | CD19 | 91,220 |
| JEKO-1 | Lymphoma | CD19 | 95,223 |
| HBL1 | Lymphoma | CD19 | 35,135 |
| Namalwa | Lymphoma | CD19 | 131,466 |
| Toledo | Lymphoma | CD19 | 155,063 |
| OCl-Ly10 | Lymphoma | CD19 | 130,995 |
| SEM | Leukemia | CD72 | 69,274 |
| RS411 | Leukemia | CD72 | 23,109 |
| JEKO-1 | Lymphoma | CD72 | 101,927 |
| HBL1 | Lymphoma | CD72 | 45,026 |
| Namalwa | Lymphoma | CD72 | 22,314 |
| Toledo | Lymphoma | CD72 | 30,723 |
| OCl-Ly10 | Lymphoma | CD72 | 121,492 |

Altogether, these studies demonstrated that CD72 is highly restricted to the B-cell compartment, and highly abundant on not only MLLr leukemias, but also on many other B-cell malignancies including other B-ALL subtypes and lymphoma samples. Therefore, CD72 is an attractive surface receptor for targeting these B cell malignancies with new immunotherapy strategies to overcome emerging resistance mechanisms to CD19 and CD22 directed CAR-T therapy.

### Example 2. Isolation of Anti-CD72 Nanobodies

### Yeast Display Enables Discovery of High-Affinity Anti-CD72 Nanobodies

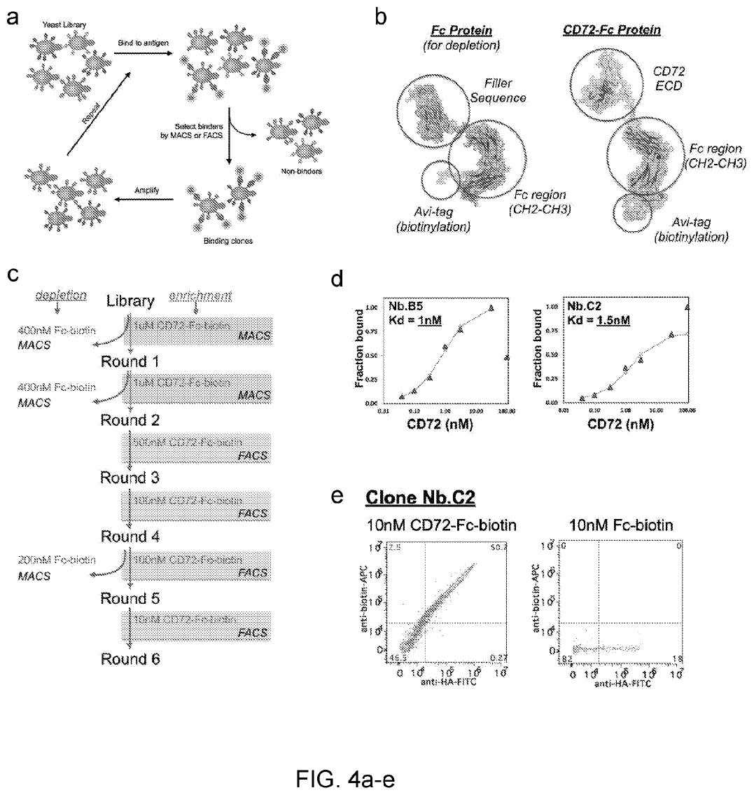

To generate CD72-specific binding reagents for use in a CAR-T cells, we employed a recently developed, fully in vitro nanobody yeast display screening platform (McMahon et al, Nat Struct Mol Biol. 1-14 (2018)) (FIG. 4a). Nanobodies are variable heavy chain-only immunoglobulins derived from camelids that, owing to their simple format, small size, and highly modular nature, are finding increasing utility in therapeutic applications. The library was initially built for enabling structural biology studies; we are the first to demonstrate its utility for immunotherapy development.

We expressed in mammalian cells a recombinant fusion protein comprised of the C-terminal extracellular domain of CD72 (aa 117-359) fused to a biotinylated human Fc domain to enable in vitro nanobody panning (FIG. 4b). After six rounds of magnetic bead and flow cytometry-based selection (FIG. 4c), >50% of the remaining nanobody-expressing yeast specifically bound CD72. Ultimately, we identified 26 unique clones. CDR3, the major binding determinant for both nanobodies and antibodies, possessed a wide range of length and sequence variability. To assess CD72 binding constants, we performed on-yeast affinity measurements. Select measured clones were estimated to possess K_{D}'s in the low-nM range for recombinant CD72 (FIG. 4d) and showed no binding to Fc-domain-only (FIG. 4e), demonstrating specificity.

### Example 3. Nanobody-Based Immunotherapies Targeting CD72 Provide Efficacious Cell Killing of B-Cell Malignancies

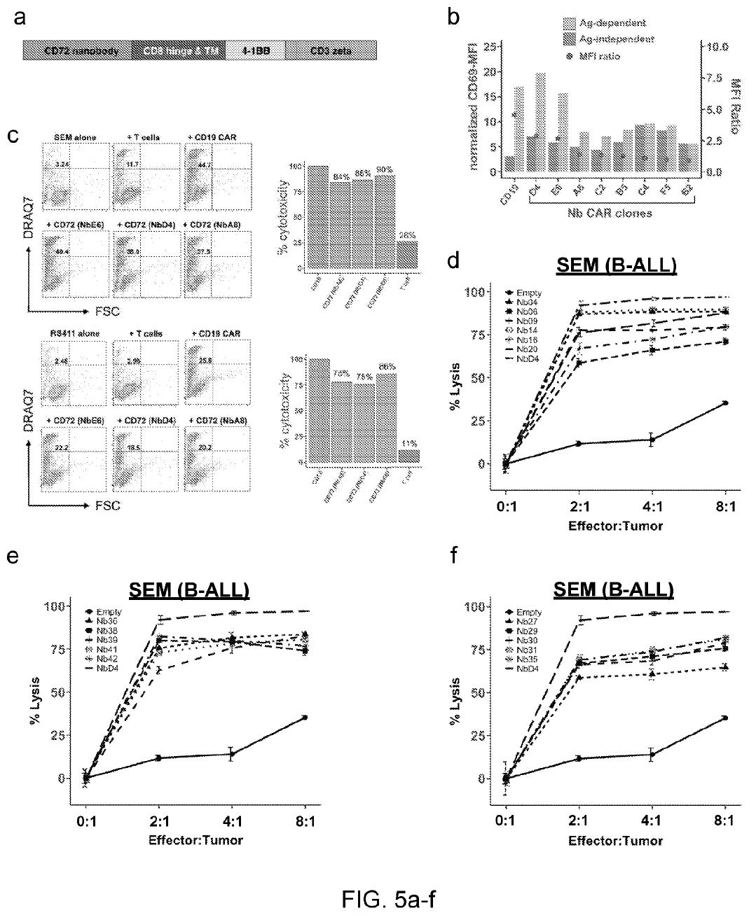

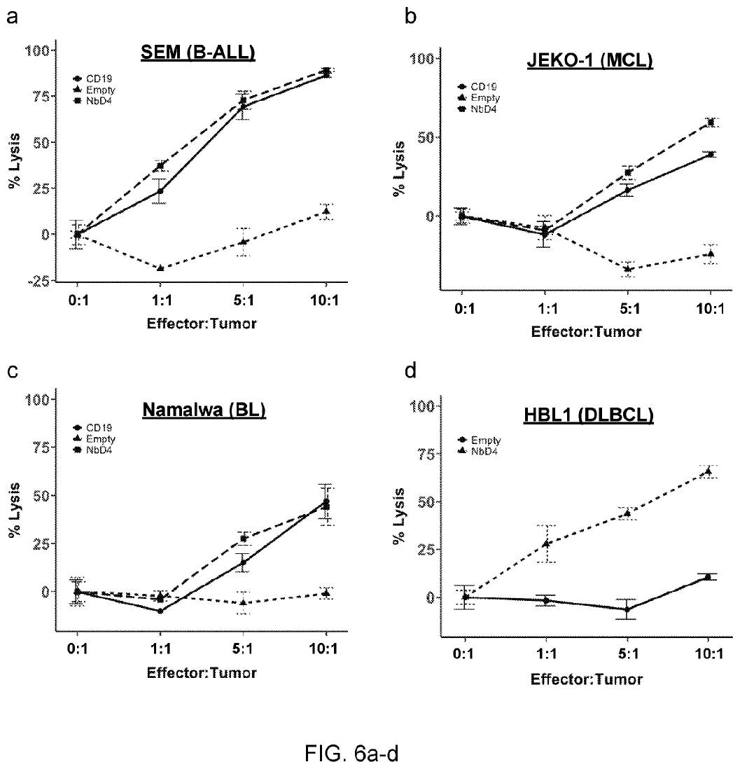

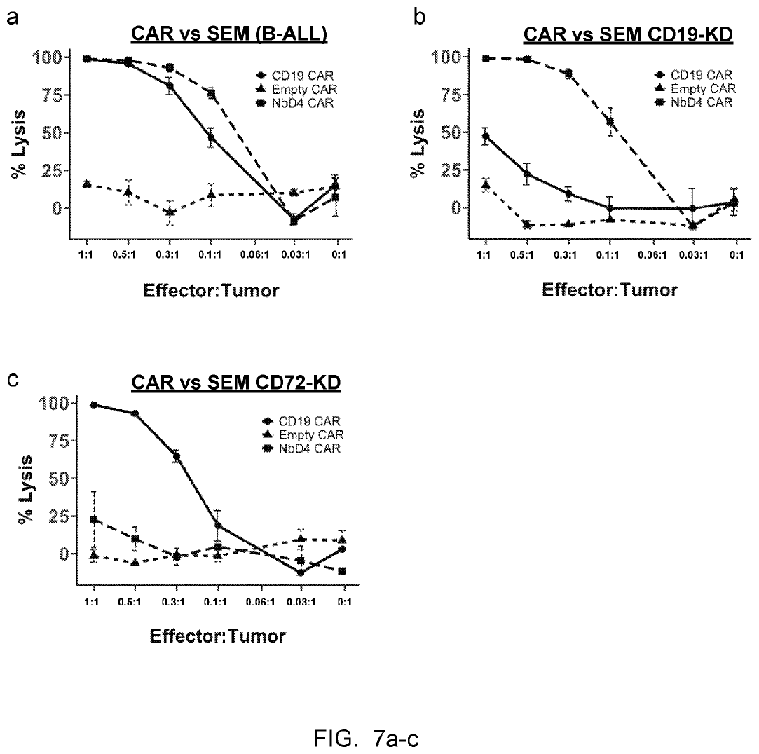

We cloned our unique nanobody sequences into a second-generation CAR-T format to screen activity in vitro. Notably, the lentiviral backbone (FIG. 5a) is identical to that used in tisangenlecleucel, an FDA-approved CD19 CAR-T. We first transduced Jurkat cells with eight nanobody-based CAR's to assess their antigen-independent and antigen-dependent activation during co-culture with either a CD72-negative cell line (AMO1, multiple myeloma) or a CD72-positive cell line (RS411, MLLr B-ALL). For all assays we used the tisangenlecleucel single chain variable fragment (scFv) CD19 binder as a positive control. At 1:1 effector:tumor (E:T) ratio, by CD69 staining we found clones D4 (nanobody sequence SEQ ID NO:6), E6 (nanobody sequence SEQ ID NO:5), and A8 (nanobody sequence SEQ ID NO:4) nanobodies sequences possessed the best antigen-dependent activation profiles in this assay (FIG. 5b). These CAR's were next transduced into normal donor T-cells, expanded using CD3/CD28 bead stimulation, sorted for CAR+ CD8+ T-cells, and screened for cytotoxicity against multiple B-cell malignancy cell lines. At 1:1 E:T at 24 h, all three nanobody CAR-T's were highly cytotoxic to both SEM and RS411, with similar efficacy to CD19 CAR-T (FIG. 5c). Evaluation of additional anti-CD72 nanobody sequences in CAR-T format revealed multiple sequences with the ability to kill SEM target cells at high E:T ratios in 8 hour co-culture assays (FIG. 5d-f). Clone Nb.D4, which possessed the best activation profile in our Jurkat assay, demonstrated superior activity compared to most nanobodies tested. Nb.D4 anti-CD72 CAR-T performed equivalently to CD19-directed CAR-T in 4 hour co-culture assays with variable E:T ratios against cell lines SEM (B-ALL), JEKO-1 (Mantle Cell Lymphoma), Namalwa (Burkitt's Lymphoma), and HBL1 (DLBCL) (FIG. 6a-d). In 48 hour co-culture assays at low effector to target ratios, CD72 (Nb.D4) CD8+ cells demonstrated potent dose-dependent cytotoxicity vs. SEM, mirroring CD19 CAR-T (FIG. 7a).

To support CD72 CAR-T as either a front-line or second-line therapy after CD19 failure, we suppressed CD19 in SEM cells using CRISPRi to generate a model of CD19 antigen escape. CD72 (Nb.D4) CAR-T was equally efficacious against CD19-negative SEM cells as parental (FIG. 7b), whereas CD19 CAR-T showed greatly diminished activity. Additionally, we knocked down CD72 and showed CD72 (Nb.D4) CAR-T had no detectable activity against these cells, whereas CD19 CAR-T retained robust killing (FIG. 7c). Thus, CD72 (Nb.D4) CAR T therapy is highly-specific and potent against CD72-bearing B-cells, and effective targeting of CD72 is independent of CD19 surface density.

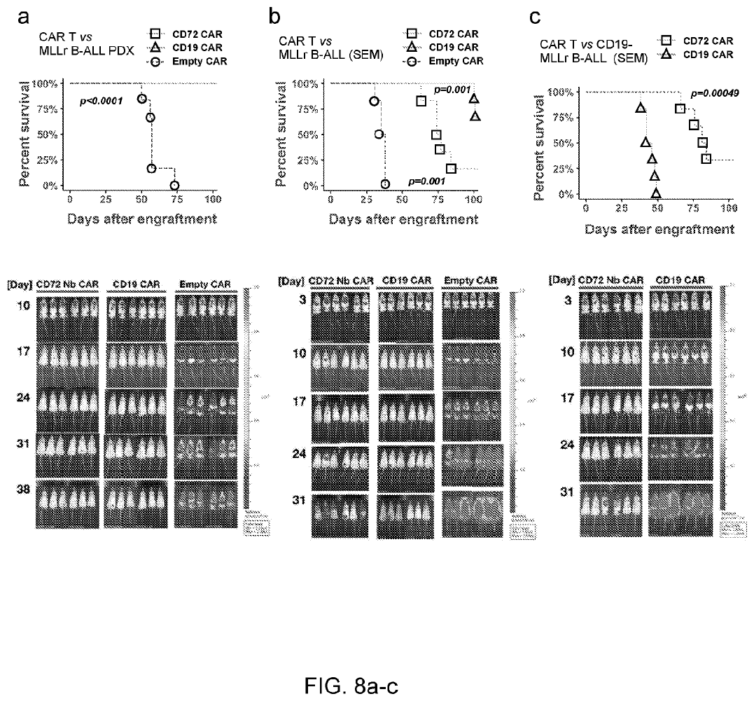

The in vivo efficacy of our CD72 CAR T was examined against an MLLr B-ALL cell line (SEM) and an MLLr B-ALL PDX in NOD scid gamma (NSG) mice. We engineered both cells to express luciferase for non-invasive bioluminescent imaging (BLI). 1e6 cells were implanted via tail vein injection and engraftment confirmed by BLI at either 3 or 10 days for SEM and PDX, respectively. Each cohort of mice (n=6 per arm) received 5e6 total CAR-T cells (a 1:1 mixture of CD4:CD8 primary T-cells) engineered with either an “empty” CAR backbone, CD72 (Nb.D4) CAR, or CD19 CAR. MLLr PDX-injected mice that received CD72 (Nb.D4) CAR-T showed a strong response and undetectable leukemic burden by BLI, comparable to CD19 CAR-T, and significantly increased survival versus the empty CAR (FIG. 8a). CD72 (Nb.D4) CAR-T performed similarly to CD19 CAR T against wild-type SEM, significantly prolonging survival compared to empty CAR (FIG. 8b). CD72 (Nb.D4) CAR-T's significantly prolonged survival against CRISPRi CD19-knockdown SEM cells in vivo compared to CD19 CAR-T (FIG. 8c).

All publications, patent applications, and accession numbers mentioned in this specification are herein incorporated by reference to the same extent as if each individual publication or patent application was specifically and individually indicated to be incorporated by reference for the material for which it is cited.

Anti-CD72 nanobody polypeptide sequences:
Nb.C2 CDR sequences are underlined
SEQ ID NO: 1
QVQLQESGGGLVQAGGSLRLSCAASGTIFDWYSMGWYRQAPGKERELVAG
IDTGANTYYADSVKGRFTISRDNAKNTVYLQMNSLKPEDTAVYYCAAHDD
GDPWHVYWGQGTQVTVSS
Nb.B5 CDR sequences are underlined
SEQ ID NO: 2
QVQLQESGGGLVQAGGSLRLSCAASGTIFPVDYMGWYRQAPGKERELVAG
INYGSNTYYADSVKGRFTISRDNAKNTVYLQMNSLKPEDTAVYYCAAWQP
EGYAVDFYHPYWGQGTQVTVSS
Nb.F5 CDR sequences are underlined
SEQ ID NO: 3
QVQLQESGGGLVQAGGSLRLSCAASGSISDRYAMGWYRQAPGKERELVAG
IAEGSNTYYADSVKGRFTISRDNAKNTVYLQMNSLKPEDTAVYYCAAHDG
WYDYWGQGTQVTVSS
Nb.A8 CDR sequences are underlined
SEQ ID NO: 4
QVQLQESGGGLVQAGGSLRLSCAASGTIFQNLDMGWYRQAPGKERELVAG
ISYGSSTYYADSVKGRFTISRDNAKNTVYLQMNSLKPEDTAVYYCAVYTY
WGQGTQVTVSS
Nb.E6 CDR sequences are underlined
SEQ ID NO: 5
QVQLQESGGGLVQAGGSLRLSCAASGSISRIGDMGWYRQAPGKERELVAA
IAAGGTTYYADSVKGRFTISRDNAKNTVYLQMNSLKPEDTAVYYCAASHE
TQPTQLVYWGQGTQVTVSS
Nb.D4 CDR sequences are underlined
SEQ ID NO: 6
QVQLQESGGGLVQAGGSLRLSCAASGTISPIDIMGWYRQAPGKEREFVAA
IALGGNTYYADSVKGRFTISRDNAKNTVYLQMNSLKPEDTAVYYCAVGYV
DKWDDSDYHTYWGQGTQVTVSS
Nb.C4 CDR sequences are underlined
SEQ ID NO: 7
QVQLQESGGGLVQAGGSLRLSCAASGSISDWYDMGWYRQAPGKEREFVAT
IANGSNTYYADSVKGRFTISRDNAKNTVYLQMNSLKPEDTAVYYCAALVG
PDDNGWYWLDYWGQGTQVTVSS
Nb.B2 CDR sequences are underlined
SEQ ID NO: 8
QVQLQESGGGLVQAGGSLRLSCAASGNISSISDMGWYRQAPGKERELVAG
IGGGANTYYADSVKGRFTISRDNAKNTVYLQMNSLKPEDTAVYYCAAHGY
WGWTHEYWGQGTQVTVSS
NB11/1-126 CDR sequences are underlined
SEQ ID NO: 9
QVQLQESGGGLVQAGGSLRLSCAASGTISSSADMGWYRQAPGKERELVAG
IDRGSNTYYADSVKGRFTISRDNAKNTVYLQMNSLKPEDTAVYYCAAEEV
GTGEDDDGADSYHGYWGQGTQVTVSS
NB27/1-126 CDR sequences are underlined
SEQ ID NO: 10
QVQLQESGGGLVQAGGSLRLSCAASGTISRDRDMGWYRQAPGKERELVAT
ISPGGTTYYADSVKGRFTISRDNAKNTVYLQMNSLKPEDTAVYYCAAYAA
VEEDDSKYYIQDFAYWGQGTQVTVSS
NB41/1-126 CDR sequences are underlined
SEQ ID NO: 11
QVQLQESGGGLVQAGGSLRLSCAASGTIFTLPDMGWYRQAPGKEREFVAG
IAGGSSTYYADSVKGRFTISRDNAKNTVYLQMNSLKPEDTAVYYCAVGYV
AESSDFYDYSNYHEYWGQGTQVTVSS
NB14/1-125 CDR sequences are underlined
SEQ ID NO: 12
QVQLQESGGGLVQAGGSLRLSCAASGNISPQHDMGWYRQAPGKERELVAT
ITQGATTYYADSVKGRFTISRDNAKNTVYLQMNSLKPEDTAVYYCAALLY
ATDPDYVYHVYHVYWGQGTQVTVSS
NB20/1-124 CDR sequences are underlined
SEQ ID NO: 13
QVQLQESGGGLVQAGGSLRLSCAASGTIFDYYDMGWYRQAPGKERELVAG
ISTGTITYYADSVKGRFTISRDNAKNTVYLQMNSLKPEDTAVYYCAAETT
SPVVGVDTLWYGYWGQGTQVTVSS
NB04/1-123 CDR sequences are underlined
SEQ ID NO: 14
QVQLQESGGGLVQAGGSLRLSCAASGSIFHYYDMGWYRQAPGKERELVAT
IDPGGTTYYADSVKGRFTISRDNAKNTVYLQMNSLKPEDTAVYYCAAYST
QRNDPETYYLDYWGQGTQVTVSS
NB06/1-122 CDR sequences are underlined
SEQ ID NO: 15
QVQLQESGGGLVQAGGSLRLSCAASGYIFQDLDMGWYRQAPGKERELVAT
ITNGGNTNYADSVKGRFTISRDNAKNTVYLQMNSLKPEDTAVYYCAAHFY
YVGYGDDEHDYWGQGTQVTVSS
NB18/1-122 CDR sequences are underlined
SEQ ID NO: 16
QVQLQESGGGLVQAGGSLRLSCAASGNISSSTDMGWYRQAPGKERELVAT
ISLGGNTYYADSVKGRFTISRDNAKNTVYLQMNSLKPEDTAVYYCAVFEK
LGLEDPLYLKYWGQGTQVTVSS
NB36/1-120 CDR sequences are underlined
SEQ ID NO: 17
QVQLQESGGGLVQAGGSLSCAASGTIFDWWDMGWYRQAPGKERELVATIS
YGGNTYYADSVKGRFTISRDNAKNTVYLQMNSLKPEDTAVYYCAVFIPGQ
WRDYYALTYWGQGTQVTVSS
NB38/1-122 CDR sequences are underlined
SEQ ID NO: 18
QVQLQESGGGLVQAGGSLRLSCAASGNISHPAHMGWYRQAPGKEREFVAA
IDDGSITYYADSVKGRFTISRDNAKNTVYLQMNSLKPEDTAVYYCAVWQE
TSVRLGIYFLYWGQGTQVTVSS
NB35/1-122 CDR sequences are underlined
SEQ ID NO: 19
QVQLQESGGGLVQAGGSLRLSCAASGSISDGDDMGWYRQAPGKEREFVAT
IDVGGNTYYADSVKGRFTISRDNAKNTVYLQMNSLKPEDTAVYYCAAAAV
DDRDGYYYLLYWGQGTQVTVSS
NB31/1-118 CDR sequences are underlined
SEQ ID NO: 20
QVQLQESGGGLVQAGGSLRLSCAASGNIFELYDMGWYRQAPGKERELVAG
ITYGANTYYADSVKGRFTISRDNAKNTVYLQMNSLKPEDTAVYYCAVHAV
NYGYLAYWGQGTQVTVSS
NB29/1-118 CDR sequences are underlined
SEQ ID NO: 21
QVQLQESGGGLVQAGGSLRLSCAASGSISAPDDMGWYRQAPGKERELVAG
IDLGGNTYYADSVKGRFTISRDNAKNTVYLQMNSLKPEDTAVYYCAAHST
EPPAYGYWGQGTQVTSS
NB16/1-118 CDR sequences are underlined
SEQ ID NO: 22
QVQLQESGGGLVQAGGSLRLSCAASGTIFWQVDMGWYRQAPGKERELVAG
ITSGTNTYYADSVKGRFTISRDNAKNTVYLQMNSLKPEDTAVYYCAAHWP
YNQTYTYWGQGTQVTVSS
NB30/1-119 CDR sequences are underlined
SEQ ID NO: 23
QVQLQESGGGLVQAGGSLRLSCAASGNIFWYAPMGWYRQAPGKERELVAS
IADGTSTNYADSVKGRFTISRDNAKNTVYLQMNSLKPEDTAVYYCAAYSE
DARDLSYWGQGTQVTVSS
NB09/1-118 CDR sequences are underlined
SEQ ID NO: 24
QVQLQESGGGLVQAGGSLRLSCAASGNIFSDFDMGWYRQAPGKERELVAG
ISVGSNTYYADSVKGRFTISRDNAKNTVYLQMNSLKPEDTAVYYCAAETV
KVDYLFYWGQGTQVTVSS
NB39/1-118 CDR sequences are underlined
SEQ ID NO: 25
QVQLQESGGGLVQAGGSLRLSCAASGTIFVSGPMGWYRQAPGKEREFVAT
ITDGASTYYADSVKGRFTISRDNAKNTVYLQMNSLKPEDTAVYYCAVADP
HDYYHHYWGQGTQVTVSS
NB42/1-117 CDR sequences are underlined
SEQ ID NO: 26
QVQLQESGGGLVQAGGSLRLSCAASGNISRYVMGWYRQAPGKERELVAGI
DVGAITYYADSVKGRFTISRDNAKNTVYLQMNSLKPEDTAVYYCAVWHYL
GYVLAYWGQGTQVTVSS

1. An isolated nanobody that specifically binds to CD72, wherein the nanobody comprises:
(1a) a CDR1 sequence comprising TISPIDI, a CDR2 sequence comprising FVAAIALGGN, and a CDR3 sequence comprising VGYVDKWDDSDYHT; or a variant thereof in which at least one of the CDRs has 1 or 2 amino acid substitutions;
(1b) a CDR1 sequence comprising TIFDWYS, a CDR2 sequence comprising LVAGIDTGAN, and a CDR3 sequence comprising AHDDGDPWHV; or a variant thereof in which at least one of the CDRs has 1 or 2 amino acid substitutions;
(1c) a CDR1 sequence comprising SISDRYA, a CDR2 sequence comprising LVAGIAEGSN, and a CDR3 sequence comprising AHDGWYD; or a variant thereof in which at least one of the CDRs has 1 or 2 amino acid substitutions;
(1d) a CDR1 sequence comprising TIFQNLD, a CDR2 sequence comprising LVAGISYGSS, and a CDR3 sequence comprising VYT; or a variant thereof in which at least one of CDR1 or CDR2 has 1 or 2 amino acid substitutions;
(1e) a CDR1 sequence comprising NISSISD, a CDR2 sequence comprising LVAGIGGGAN, and a CDR3 sequence comprising AHGYWGWTHE; or a variant thereof in which at least one of the CDRs has 1 or 2 amino acid substitutions;
(1f) a CDR1 sequence comprising TIFPVDY, a CDR2 sequence comprising LVAGINYGSN, and a CDR3 sequence comprising AWQPEGYAVDFYHP; or a variant thereof in which at least one of the CDRs has 1 or 2 amino acid substitutions;
(1g) a CDR1 sequence comprising SISDWYD, a CDR2 sequence comprising FVATIANGSN, and a CDR3 sequence comprising ALVGPDDNGWYWLD; or a variant thereof in which at least one of the CDRs has 1 or 2 amino acid substitutions;
(1h) a CDR1 sequence comprising SISRIGD, a CDR2 sequence comprising LVAAIAAGGT, and a CDR3 sequence comprising ASHETQPTQLV; or a variant thereof in which at least one of the CDRs has 1 or 2 amino acid substitutions;
(2a) a CDR1 sequence comprising TISSSAD, a CDR2 sequence comprising LVAGIDRGSN, and a CDR3 sequence comprising AEEVGTGEDDDGADSYHG; or a variant thereof in which at least one of the CDRs has 1 or 2 amino acid substitutions;
(2b) a CDR1 sequence comprising TISRDRD, a CDR2 sequence comprising LVATISPGGT, and a CDR3 sequence comprising AYAAVEEDDSKYYIQDFA; or a variant thereof in which at least one of the CDRs has 1 or 2 amino acid substitutions;
(2c) a CDR1 sequence comprising TIFTLPD, a CDR2 sequence comprising VAGIAGGSS, and a CDR3 sequence comprising VGYVAESSDFYDYSNYHE; or a variant thereof in which at least one of the CDRs has 1 or 2 amino acid substitutions;
(2d) a CDR1 sequence comprising NISPQHD, a CDR2 sequence comprising LVATITQGAT, and a CDR3 sequence comprising ALLYATDPDYVYHVYHV; or a variant thereof in which at least one of the CDRs has 1 or 2 amino acid substitutions;
(2e) a CDR1 sequence comprising TIFDYYD, a CDR2 sequence comprising LVAGISTGTI, and a CDR3 sequence comprising AETTSPVVGVDTLWYG; or a variant thereof in which at least one of the CDRs has 1 or 2 amino acid substitutions;
(2f) a CDR1 sequence comprising SIFHYYD, a CDR2 sequence comprising LVATIDPGGT, and a CDR3 sequence comprising AYSTQRNDPETYYLD; or a variant thereof in which at least one of the CDRs has 1 or 2 amino acid substitutions;
(2g) a CDR1 sequence comprising YIFQDLD, a CDR2 sequence comprising LVATITNGGN, and a CDR3 sequence comprising AHFYYVGYGDDEHD; or a variant thereof in which at least one of the CDRs has 1 or 2 amino acid substitutions;
(2h) a CDR1 sequence comprising NISSSTD, a CDR2 sequence comprising LVATISLGGN, and a CDR3 sequence comprising VFEKLGLEDPLYLK; or a variant thereof in which at least one of the CDRs has 1 or 2 amino acid substitutions;
(2i) a CDR1 sequence comprising TIFDWWD, a CDR2 sequence comprising LVATISYGGN, and a CDR3 sequence comprising VFIPGQWRDYYALT; or a variant thereof in which at least one of the CDRs has 1 or 2 amino acid substitutions;
(2j) a CDR1 sequence comprising NISHPAH, CDR2 sequence comprising FVAAIDDGSI, a CDR3 sequence comprising VWQETSVRLGIYFL; a variant thereof in which at least one of the CDRs has 1 or 2 amino acid substitutions;
(2k) a CDR1 sequence comprising SISDGDD, a CDR2 sequence comprising FVATIDVGGN, and a CDR3 sequence comprising AAAVDDRDGYYYLL; or a variant thereof in which at least one of the CDRs has 1 or 2 amino acid substitutions;
(2l) a CDR1 sequence comprising NIFELYD, a CDR2 sequence comprising LVAGITYGAN, and a CDR3 sequence comprising VHAVNYGYLA; or a variant thereof in which at least one of the CDRs has 1 or 2 amino acid substitutions;
(2m) a CDR1 sequence comprising SISAPDD, a CDR2 sequence comprising LVAGIDLGGN, and a CDR3 sequence comprising AHSTEPPAYG; or a variant thereof in which at least one of the CDRs has 1 or 2 amino acid substitutions;
(2n) a CDR1 sequence comprising TIFWQVD, a CDR2 sequence comprising LVAGITSGTN, and a CDR3 sequence comprising AHWPYNQTYT; or a variant thereof in which at least one of the CDRs has 1 or 2 amino acid substitutions;
(2o) a CDR1 sequence comprising NIFWYAP, a CDR2 sequence comprising LVASIADGTS, and a CDR3 sequence comprising AYSEDARDLS; or a variant thereof in which at least one of the CDRs has 1 or 2 amino acid substitutions;
(2p) a CDR1 sequence comprising NIFSDFD, a CDR2 sequence comprising LVAGISVGSN, and a CDR3 sequence comprising AETVKVDYLF; or a variant thereof in which at least one of the CDRs has 1 or 2 amino acid substitutions;
(2q) a CDR1 sequence comprising TIFVSGP, a CDR2 sequence comprising FVATITDGAS, and a CDR3 sequence comprising VADPHDYYHH; or a variant thereof in which at least one of the CDRs has 1 or 2 amino acid substitutions; or
(2r) a CDR1 sequence comprising NISRYV, a CDR2 sequence comprising LVAGIDVGAI, and a CDR3 sequence comprising VWHYLGYVLA; or a variant thereof in which at least one of the CDRs has 1 or 2 amino acid substitutions.

2. The isolated nanobody of claim 1, comprising:
(1a) a CDR1 sequence comprising TISPIDI, a CDR2 sequence comprising FVAAIALGGN, and a CDR3 sequence comprising VGYVDKWDDSDYHT; or a variant thereof in which at least one of the CDRs has 1 or 2 amino acid substitutions;
(1b) a CDR1 sequence comprising TIFDWYS, a CDR2 sequence comprising LVAGIDTGAN, and a CDR3 sequence comprising AHDDGDPWHV; or a variant thereof in which at least one of the CDRs has 1 or 2 amino acid substitutions;
(1c) a CDR1 sequence comprising SISDRYA, a CDR2 sequence comprising LVAGIAEGSN, and a CDR3 sequence comprising AHDGWYD; or a variant thereof in which at least one of the CDRs has 1 or 2 amino acid substitutions;
(1d) a CDR1 sequence comprising TIFQNLD, a CDR2 sequence comprising LVAGISYGSS, and a CDR3 sequence comprising VYT; or a variant thereof in which at least one of CDR1 or CDR2 has 1 or 2 amino acid substitutions;
(1e) a CDR1 sequence comprising NISSISD, a CDR2 sequence comprising LVAGIGGGAN, and a CDR3 sequence comprising AHGYWGWTHE; or a variant thereof in which at least one of the CDRs has 1 or 2 amino acid substitutions;
(1f) a CDR1 sequence comprising TIFPVDY, a CDR2 sequence comprising LVAGINYGSN, and a CDR3 sequence comprising AWQPEGYAVDFYHP; or a variant thereof in which at least one of the CDRs has 1 or 2 amino acid substitutions;
(1g) a CDR1 sequence comprising SISDWYD, a CDR2 sequence comprising FVATIANGSN, and a CDR3 sequence comprising ALVGPDDNGWYWLD; or a variant thereof in which at least one of the CDRs has 1 or 2 amino acid substitutions; or
(1h) a CDR1 sequence comprising SISRIGD, a CDR2 sequence comprising LVAAIAAGGT, and a CDR3 sequence comprising ASHETQPTQLV; or a variant thereof in which at least one of the CDRs has 1 or 2 amino acid substitutions.

3.-4. (canceled)

5. The isolated nanobody of claim 1, wherein the framework: (i) has at least 80% identity to a human antibody heavy chain framework; (ii) is a VH3 family member; or (iii) has at least 80% identity to a framework comprising an FR1 sequence QVQLQESGGGLVQAGGSLRLSCAAS, an FR2 sequence MGWYRQAPGKERE, an FR3 sequence TYYADSVKGRFTISRDNAKNTVYLQMNSLKPEDTAVYYCA, and an FR4 sequence YWGQGTQVTVSS.

6.-7. (canceled)

8. A bispecific or multispecific antibody comprising a nanobody of claim 1.

9. A chimeric antigen receptor (CAR) comprising an antigen binding domain, a transmembrane domain, and an intracellular domain comprising a costimulatory domain and/or a primary signaling domain, wherein the antigen binding domain comprises a nanobody of claim 1.

10. (canceled)

11. The CAR of claim 9, wherein the antigen binding domain comprises at two, three or four nanobodies selected from the group consisting of:
(a) a nanobody comprising a CDR1 sequence comprising TIFDWYS, a CDR2 sequence comprising LVAGIDTGAN, and a CDR3 sequence comprising AHDDGDPWHV;
(b) a nanobody comprising a CDR1 sequence comprising SISDRYA, a CDR2 sequence comprising LVAGIAEGSN, and a CDR3 sequence comprising AHDGWYD;
(c) a nanobody comprising a CDR1 sequence comprising TIFQNLD, a CDR2 sequence comprising LVAGISYGSS, and a CDR3 sequence comprising VYT;
(d) a nanobody comprising a CDR1 sequence comprising NISSISD, a CDR2 sequence comprising LVAGIGGGAN, and a CDR3 sequence comprising AHGYWGWTHE;
(e) a nanobody comprising a CDR1 sequence comprising TIFPVDY, a CDR2 sequence comprising LVAGINYGSN, and a CDR3 sequence comprising AWQPEGYAVDFYHP;
(f) a nanobody comprising a CDR1 sequence comprising SISDWYD, a CDR2 sequence comprising FVATIANGSN, and a CDR3 sequence comprising ALVGPDDNGWYWLD;
(g) a nanobody comprising a CDR1 sequence comprising TISPIDI, a CDR2 sequence comprising FVAAIALGGN, and a CDR3 sequence comprising VGYVDKWDDSDYHT; and
(h) a nanobody comprising a CDR1 sequence comprising SISRIGD, a CDR2 sequence comprising LVAAIAAGGT, and a CDR3 sequence comprising ASHETQPTQLV.

12.-13. (canceled)

14. The CAR of claim 9, wherein the CAR is a standard CAR, a split CAR, an off-switch CAR, an on-switch CAR, a first-generation CAR, a second-generation CAR, a third-generation CAR, or a fourth-generation CAR.

15. An immune effector cell comprising a CAR of claim 9.

16. The immune effector cell of claim 15, wherein the cell is a T lymphocyte or a natural killer (NK) cell.

17. A method of treating a hematological malignancy that comprises malignant B cells that express CD72 or a malignancy that comprises malignant myeloid cells that express CD72, the method comprising administering a plurality of immune effector cells of claim 15 to a subject that has the hematological malignancy.

18. The method of claim 17, wherein the plurality of immune effector cells comprises allogeneic cells or comprises autologous cells.

19. (canceled)

20. The method of claim 17, wherein the hematological malignancy is a B-cell leukemia, a non-Hodgkin's lymphoma, or a multiple myeloma.

21. The method of claim 20, wherein the hematological malignancy is a B cell leukemia and the B-cell leukemia is chronic lymphocytic leukemia or mixed-lineage leukemia (MLL).

22.-24. (canceled)

25. The method of claim 17, wherein the subject is a human.

26. A polynucleotide encoding a CAR of claim 9.

27. A vector comprising the polynucleotide of claim 26.

28.-29. (canceled)

30. An immune effector cell comprising a polynucleotide of claim 27.

31. The immune effector cell of claim 30, wherein the cell is a T lymphocyte or NK cell.

32. A host cell comprising the polynucleotide of claim 26.

33.-34. (canceled)

35. A nucleic acid encoding a nanobody of claim 1.

36. An expression vector or host cell comprising the nucleic acid of claim 35.

37.-38. (canceled)
